(19) United States
(12) Patent Application Publication
(10) Pub. No.: US 2016/0095958 A1
(43) Pub. Date: Apr. 7, 2016

(54) BONE REGENERATION USING STROMAL VASCULAR FRACTION, PLATELET-DERIVED GROWTH FACTOR-RICH HYDROGEL, THREE-DIMENSIONAL PRINTED POLY-EPSILON-CAPROLACTONE SCAFFOLDS

(71) Applicant: THE JOHNS HOPKINS UNIVERSITY, Baltimore, MD (US)

(72) Inventors: WARREN GRAYSON, REISTERSTOWN, MD (US); COLIN COOK, BALTIMORE, MD (US); BEN P.J. HUNG, BALTIMORE, MD (US); PINAR HURI, BALTIMORE, MD (US); DAPHNE L. HUTTON, BALTIMORE, MD (US); JOSHUA TEMPLE, BALTIMORE, MD (US)

(73) Assignee: THE JOHNS HOPKINS UNIVERSITY, BALTIMORE, MD (US)

(21) Appl. No.: 14/893,286

(22) PCT Filed: May 28, 2014
(86) PCT No.: PCT/US2014/039767

§ 371 Date: Nov. 23, 2015

Related U.S. Application Data
(60) Provisional application No. 61/828,114, filed on May 28, 2013.

(51) Int. Cl. A61L 27/38; A61L 27/56; A61L 27/22; A61L 27/54; A61L 27/18; A61L 27/58; A61L 27/52

(52) U.S. Cl. CPC A61L 27/3847; A61L 27/58; A61L 27/56; A61L 27/52; A61L 27/54; A61L 27/3821; A61L 27/3808; A61L 27/3834; A61L 27/18; A61L 27/225; A61L 27/3886; A61L 27/3895; A61L 2300/414; A61L 2430/02; A61L 2300/64; A61L 2300/62; A61L 2300/604; A61L 2300/412; A61L 2300/252

(57) ABSTRACT

The presently disclosed subject matter focuses on recapitulating the heterotypic interactions needed to maximize the co-development of vasculature and bone. More particularly, the presently disclosed subject matter explores the potential of cellular aggregation and temporal presentation of factors to induce the cell-cell signaling events required to stimulate ASCs to self-organize into vascularized bone. Further, exogenous PDGF-BB synergizes complex tissue formation in ASC cultures by enhancing vascular stability and osteogenic differentiation. The presently disclosed approach provides a robust protocol to engineer vascularized bone with ASCs in vitro.

### CROSS-REFERENCE TO RELATED APPLICATION

This application claims the benefit of U.S. Provisional Application No. 61/828,114, filed May 28, 2013; which is incorporated herein by reference in its entirety.

### BACKGROUND

Tissue engineering approaches to generate vascularized bone grafts could potentially revolutionize treatment of massive bone loss due to traumatic injuries, cancer, and congenital defects. Vasculature is essential to the long-term functional outcomes of a large bone graft to ensure sufficient nutrient delivery and post-implantation viability. While vasculature plays a necessary and intimate role in bone development, inducing the formation of vascularized bone tissues in vitro remains a challenge because the factors that promote the vascular lineage and the osteogenic lineage may be detrimental to the other. Quarto N., Longaker M. T. Tissue Eng 12, 1405-18, 2006; Meury T., et al., J Cell Biochem 98, 992-1006, 2006; and Hori Y., et al., Br J Pharmacol 118, 1584-91, 1996.

This incompatibility has led researchers to develop various methods to encourage vasculature to form concurrently or sequentially with osteogenic differentiation, including, but not limited to: precise dosing regimens of induction factors, Correia C., et al., J Tissue Eng Regen Med 2012; Correia C., et al., PLoS One 6, e28352, 2011; and Tsigkou O., et al., Proc Natl Acad Sci USA 107, 3311-6, 2010; various cocktail media, Rouwkema J., et al., Tissue Eng 12, 2685-93, 2006; Liu Y., et al., Stem Cells 30, 1911-24, 2012; and pre-treating multiple cell types separately followed by recombination in a unified graft. Correia C., et al., PLoS One 6, e28352, 2011; Tsigkou O., et al., Proc Natl Acad Sci USA 107, 3311-6, 2010; Usami K., et al., J Biomed Mater Res A 90, 730-41, 2009; Koob S., et al., Tissue Eng Part A 17, 311-21, 2011; and Nishi M., et al., J Biomed Mater Res A 2012. Yet each of these approaches relies on mitigating competing factors, which results in an imbalance between those competing factors and leads to sub-optimal results for one or both of the tissue components.

Adipose-derived stromal/stem cells (ASCs) are an ideal, clinically relevant cell source to supply both the osteogenic and vascular components of a vascularized bone graft. In addition to their well-studied osteogenic capacity, Gimble J., Guilak F., Cytotherapy 5, 362-9, 2003; Zuk P. A., et al., Mol Biol Cell 13, 4279-95, 2002; ASC cultures also have been shown to give rise to endothelial cells, Cao Y., et al., Biochem Biophys Res Commun 332, 370-9, 2005; Zonari A., et al., PLoS One 7, e35422, 2012; and lumen-containing vessels. Hutton D. L., et al., Tissue Eng Part A 18, 1729-40, 2012; Miranville A., et al., Circulation 110, 349-55, 2004; Planat-Benard V., et al., Circulation 109, 656-63, 2004.

The ability of ASCs to undergo direct differentiation to endothelial cells, however, remains contentious. Boquest A. C., et al, Stem Cells 25, 852-61, 2007. Recent findings suggest that these vessels may arise due to a minute sub-population of residual committed endothelial cell progenitors that undergo extensive proliferation and self-assembly to form vascular networks. Hutton D. L., et al., Tissue Eng Part A 18, 1729-40, 2012; Koh Y. J., et al., Arterioscler Thromb Vasc Biol 31, 1141-50, 2011. Recently, a few groups have shown promise in the potential of using ASCs, Correia C., et al., J Tissue Eng Regen Med 2012; Gardin C., et al., Stem Cells Dev 21, 767-77, 2012, or fresh stromal vascular fraction (SVF) cells, Guven S., et al., Biomaterials 32, 5801-9, 2011; Müller A. M., et al., Eur Cell Mater 19, 127-35, 2010, to form vascularized bone.

Robust vascular network development coupled with dense mineral deposition, however, has yet to be demonstrated, which may limit the extent of integration and functional outcome of the tissue. This problem is a fundamental challenge for all approaches toward engineering a vascularized bone graft, including those approaches that involve the combination of osteoblasts or mesenchymal stem cells (MSCs) with a separate endothelial cell source. Liu Y., et al., J Tissue Eng Regen Med 2012.

Prior studies showed that vascular assembly of ASCs depends on heterotypic cell-cell interactions, specifically through dense clustering of cells and endogenous platelet derived growth factor (PDGF) signaling. PDGF is one of several growth factors or proteins that regulate cell growth and division and it plays a significant role in blood vessel formation (angiogenesis), i.e., the growth of blood vessels from already-existing blood vessel tissue. PDGF is a dimeric glycoprotein comprising two A (-AA) chains, two B (-BB) chains, or a combination thereof (-AB).

This endogenous behavior is reminiscent of what occurs in native tissues, as proliferating endothelial cells in nascent vessels secrete PDGF-BB to recruit pericytes for vascular maturation and stabilization. Armulik A., et al., Circ Res 97, 512-23, 2005; Bergers G., Song S., Neuro Oncol 7, 452-64, 2005. PDGF-BB also is a major factor secreted by activated platelets to stimulate repair mechanisms in wounded tissues, such as bone. Caplan A. I., Correa D., J Orthop Res 29, 1795-803, 2011; Solheim E., Int Orthop 22, 410-6, 1998; Andrew J. G., et al., Bone 16, 455-60, 1995.

A number of studies have demonstrated that in vivo administration of exogenous PDGF-BB significantly enhances bone formation. Graham S., et al., Expert Opin Investig Drugs 18, 1633-54, 2009; Park Y. J., et al., Biomaterials 21, 153-9, 2000; and Nash T. J., et al., Bone 15, 203-8, 1994. Yet, in vitro studies with MSCs have demonstrated that, while exogenous PDGF-BB induces greater proliferation, it has no effect on, Chaudhary L. R., et al., Bone 34, 402-11, 2004; Kumar A., Salimath B. P., Stark G. B., Finkenzeller G., Tissue Eng Part A 16, 983-93, 2010; and Tanaka H., Liang C. T., J Cell Physiol 164, 367-75, 1995, or may even be inhibitory to, Gruber R., et al., Platelets 15, 29-35, 2004; Tokunaga A., et al., J Bone Miner Res 23, 1519-28, 2008, osteogenic differentiation. With regard to ASCs, it has been shown that during osteogenic differentiation their expression of PDGF receptor β (PDGFR-β) is upregulated. Lee J., et al., J Craniofac Surg 21, 1136-41, 2010. The ability of PDGF-BB signaling to directly enhance the osteogenic capabilities of ASCs, however, is not known. New studies showing direct comparisons between ASCs and MSCs confirm this distinction between the two cell populations (FIG. 1).

### SUMMARY

The presently disclosed subject matter demonstrates that platelet derived growth factor and spatiotemporal cues induce development of vascularized bone tissue by autologous cells derived from bone marrow or adipose tissue. Accordingly, the presently disclosed subject matter provides a clinically translatable approach to induce robust formation of both vasculature and bone within a unified graft using a single autologous cell source.

In some aspects, the presently disclosed subject matter provides a biodegradable scaffold for regenerating bone tissue, the scaffold configured to form a porous, three-dimensional (3D) network of interconnected void spaces, wherein the porous, three-dimensional network further comprise a hydrogel, and wherein the hydrogel comprises one or more cells encapsulated therein and one or more growth factors capable of promoting regeneration of bone tissue.

In other aspects, the presently disclosed subject matter provides a method for preparing a composite for promoting regeneration of bone tissue, the method comprising: (a) providing a hydrogel; (b) encapsulating one or more cells in the hydrogel; (c) culturing the encapsulated cells for a first period of time in a vascular medium (VM); (d) waiting for a second period of time, then culturing the encapsulated cells in an osteogenic medium (OM); and (e) adding one or more growth factors to the hydrogel during at least one period of time while the encapsulated cells are cultured in either the VM or the OM.

In yet other aspects, the presently disclosed subject matter provides a method for treating a bone defect, the method comprising contacting the bone defect with a biodegradable scaffold for regenerating bone tissue, the scaffold configured to form a porous, three-dimensional (3D) network of interconnected void spaces, wherein the porous, three-dimensional network further comprise a hydrogel, and wherein the hydrogel comprises one or more cells encapsulated therein and one or more growth factors capable of promoting regeneration of bone tissue.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows that ASCs and MSCs exhibit a differential osteogenic response to exogenous PDGF (Osteogenic+);

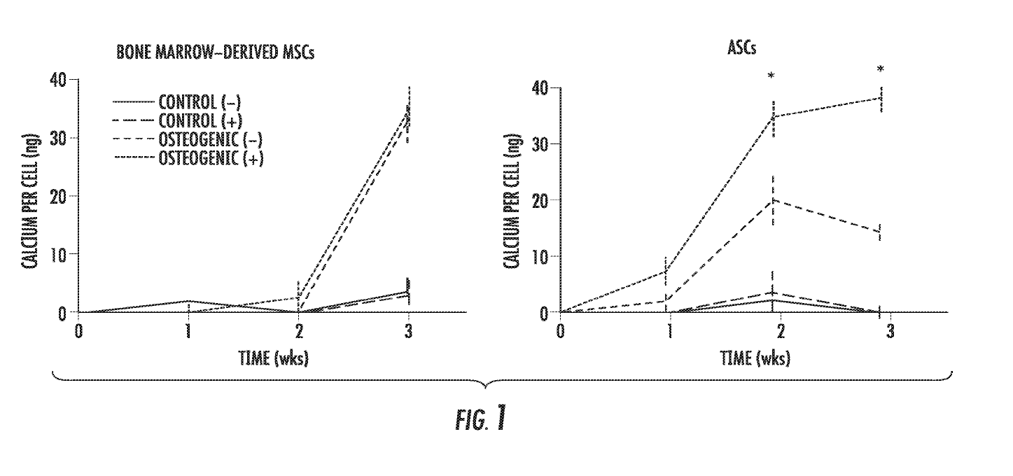

FIGS. 2A-2E show the effects of dexamethasone and FGF-2 on each lineage: (A,B) osteogenic cultures yielded significantly greater calcium deposition with the addition of 1 ng/mL FGF-2 and with dexamethasone concentrations ranging between 0 nM to 10 nM. (C) In vascular cultures, dexamethasone substantially inhibited vessel network growth (D) while increasing pericyte coverage (E) in a dose-dependent manner. Addition of FGF-2 increased vessel width (C), and induced a slight increase in vessel network length and pericyte coverage. Scale bar=500 μm. Significance indicated by brackets in (B,D,E) as *P<0.05, **P<0.01, or ***P<0.001. Significance indicated in (B) as ##P<0.01 or ###P<0.001 versus D100-F0, and all OM groups were significantly greater (P<0.001) than control. Abbreviations: X nM dexamethasone (“DX”); Y ng/mL FGF-2 (“FY”);

FIG. 3 shows the phenotypic distribution of initial cell population. ASCs at passage 2 were primarily positive for mesenchymal markers CD73 and CD105 and predominantly negative for markers of the endothelial lineage and the pericyte marker, alpha-smooth muscle actin (αSMA);

FIGS. 4A-4F show the effects of cellular aggregation on vascular morphogenesis: (A) ASCs were encapsulated in fibrin gel as monodispersed cells or as spheroid aggregates (“sph”) of different sizes; (B) vascular networks developed after 14 days of culture in vascular medium, staining positively for CD31 (green), αSMA (red), and laminin (blue). Compared to monodispersed cultures vascular networks derived from spheroids were longer (C), more interconnected (D), and displayed greater coverage by pericytes (E) and laminin (F). Scale bars=500 μm. Significance indicated as *P<0.05, **P<0.01, or ***P<0.001 versus monodispersed;

FIGS. 5A-5E show sequential addition of factors: (A) fibrin-encapsulated spheroids were cultured for 0, 4, 8, or 12 days in vascular medium (VM) before switching to osteogenic medium (OM). All samples were cultured for 12 days. (Scale bar=500 μm). Total vascular network length (B) and laminin coverage (D) were significantly higher when the transition to OM was delayed by several days whereas αSMA staining intensity increased substantially with longer culture periods in OM (C). (B-D) Significance indicated as *P<0.05, **P<0.01, or ***P<0.001 versus 0 days. (E) The step-wise protocol resulted in early mineral deposits being deposited in the vicinity of established vessels (arrows). Scale bar=50 μm;

FIGS. 6A-6E show the independent effects of exogenous PDGF-BB on each lineage. Exogenous PDGF-BB increased mineral deposition in osteogenic cultures, as indicated by Alizarin Red S staining (A) and calcium quantification (B). Conversely, blocking endogenous PDGF receptor signaling with AG1295 greatly reduced overall mineral deposition (A), but not on a per-cell basis (B). In vascular cultures, vessel network length was minimally affected by PDGF signaling (C,D), whereas pericyte coverage was substantially reduced in the presence of exogenous PDGF-BB (C,E). Scale bar=500 μm. Significance indicated by brackets in (B,D,E) as *P<0.05, **P<0.01, or ***P<0.001. Significance indicated in (B) as ###P<0.001 versus PDGF groups, and all OM groups were significantly greater (P<0.001) than control;

FIGS. 7A-7H show the combined effects of exogenous PDGF-BB on vascularized bone. Encapsulated spheroids were cultured for 8 days in VM, followed by 12 days of OM with 0 ng/mL, 2 ng/mL, or 20 ng/mL of PDGF-BB added throughout the entire culture period. Total vessel length (A,E), osteocalcin production (C,G), and calcium deposition (H) increased while αSMA staining intensity (B,F) decreased with increasing PDGF-BB concentration. Scale bar=500 μm. Significance indicated as *P<0.05, **P<0.01, or ***P<0.001 versus 20 ng/mL PDGF-BB;

FIGS. 8A-8G show a direct comparison of PDGF-BB versus BMP-2 on vascularized bone. Encapsulated spheroids were cultured for 8 days in VM, followed by 12 days of OM with the addition of 20 ng/mL of either PDGF-BB or BMP-2. Exogenous BMP-2 induced no significant changes in either vascular network length (A,E) or calcium content (H) versus the control group (“VM-OM”), whereas PDGF-BB was substantially greater than both. (B,F) αSMA staining intensity was much greater with BMP-2 versus PDGF-BB, but only slightly greater than control. (C,G) Osteocalcin staining intensity was similar between PDGF-BB and BMP-2 groups. Scale bar=500 μm. Significance indicated as *P<0.05, **P<0.01, or ***P<0.001 versus 20 ng/mL PDGF-BB;

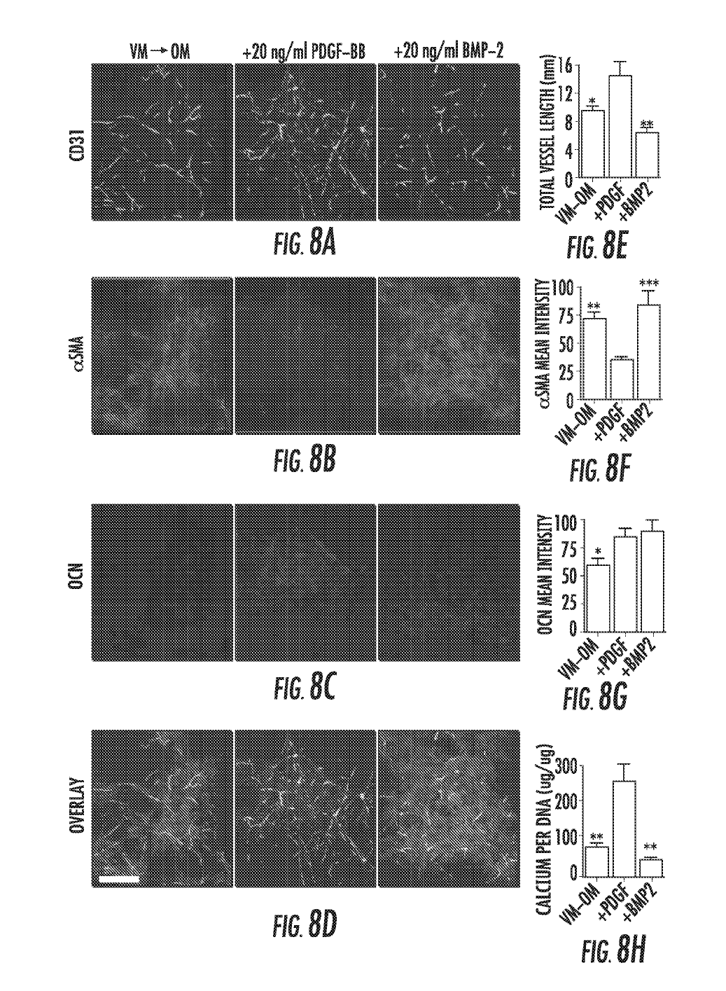

FIG. 9 shows the osteogenic differentiation of ASCs in the absence (Control) or presence of pro- and anti-inflammatory cytokines. In each case, the cytokine was provided for the first two days of culture and removed for the remaining 19 days. The samples are stained for calcium deposition (red) which is indicative of new bone formation;

FIGS. 10A-10E show scaffold characterization: (A) cuboidal PCL scaffolds of varying porosities were imaged top-down using a stereomicroscope at 1× magnification. From the scaffold parameters, an ideal pore geometry was created using MATLAB; (B) the scaffold images were converted to binary and thresholded at a value of 75% to clearly isolate the pores; (C) a normalized cross correlation was performed between the scaffold and the theoretically ideal pore, generating a heat map of areas of strongest correlation. This heat map was converted into a quantitative value termed the correlation factor; (D) this approach was used to quantify the quality of scaffolds at varying temperatures; and (E) feed rates;

FIG. 11 shows scanning electron microscopy: (Upper row) stereomicroscope images of 15×15×5 mm scaffolds with infill densities ranging from 20 to 80%; (Middle row) SEM images taken at 25× magnification demonstrate the uniformity of the pores and fiber widths. Scale bar=1 mm; and (Bottom row) SEM images at 55× magnification illustrate surfaces of fibers. Scale bar=200 μm;

FIG. 12 shows infill density versus pore size. Relationship between infill density and the actual pore size, measured from the SEM images. The best fit line is an exponential decay regression, P=4.14e-^{0.04d} where P is the pore size and d is the infill density;

FIGS. 13A-13D show cell seeding distribution. DAPI staining of scaffold cross-sections shows ASC aggregate distribution on scaffolds with various infill densities: (A) 20%; (B) 30%; (C) 40%; and (D) 50%. Dotted yellow lines represent scaffold struts. Scale bar represents 1 mm;

FIGS. 14A-14D show in vitro vascularization and mineralization. ASC aggregates were seeded with fibrin gel into PCL scaffolds and cultured in vascular or osteogenic medium for 14 days: (A) in vascular conditions, extensive vascular networks form within the pore spaces of the scaffold; (B) vessels wrap closely around PCL fibers. Colors: CD31 (green, endothelial), αSMA (red, perivascular), NG2 (blue, perivascular); and (C, D) in osteogenic conditions, mineral (black) is deposited throughout the pore spaces and along PCL fibers. Scale bars=250 μm (A, B), 100 μm (C, D);

FIGS. 15A-15I show in vivo vascularization. PCL scaffolds were implanted in rat dorsal subcutaneous pockets for 7 days to assess vascular infiltration. Grafts were: acellular (fibrin only), freshly seeded with cells (uncultured cells in fibrin), or prevascularized (cells in fibrin, cultured for 18 days in vascular medium): (A-C) hematoxylin and eosin staining demonstrates the cellular density of each tissue, where acellular scaffolds (A) still contain sparsely infiltrated remnants of fibrin denoted by an asterisk (*) in the center; (D-F) immunohistochemical staining for CD31 (green, endothelial marker) and αSMA (red, pericyte marker) shows the extent of vascularity, where cell-seeded scaffolds (E, F) show higher density in the center than those without cells; and (D, G-I) magnified views of the boxed areas in (D-F) demonstrate differences in the number of lumen-containing (arrowhead), pericyte-stabilized vessels within the center of the graft. Scale bars=500 μm (A-F), 200 μm (G-I); and

FIG. 16 shows anatomically shaped scaffolds. Left: isolated 3D geometries of the maxilla (top) and mandible (bottom). Right: 3D-printed, porous PCL scaffolds at 40% infill density.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Bone Regeneration Using Stromal Vascular Fraction, Platelet-Derived Growth Factor-Rich Hydrogel, Three-Dimensional (3D) Printed Poly-ε-Caprolactone Scaffolds

Vasculature is essential to the functional integration of a tissue-engineered bone graft to enable sufficient nutrient delivery and viability after implantation. Native bone and vasculature develop through intimately coupled, tightly regulated spatiotemporal cell-cell signaling. The complexity of these developmental processes has been a challenge for tissue engineers to recapitulate, resulting in poor co-development of both bone and vasculature within a unified graft.

To address this challenge, the presently disclosed methods provide cultured adipose-derived stromal/stem cells (ASCs), a clinically relevant, single cell source that has been previously investigated for its ability to give rise to vascularized bone grafts, and investigated the effects of initial spatial organization of cells, the temporal addition of growth factors, and the presence of exogenous platelet-derived growth factor-BB (PDGF-BB) on the co-development of bone and vascular tissue structures.

More particularly, human ASCs were aggregated into multicellular spheroids prior to encapsulation and subsequent outgrowth in fibrin gels. In some embodiments, cellular aggregation was found to substantially increase vascular network density, interconnectivity, and pericyte coverage as compared to monodispersed cultures. To form robust vessel networks, ASCs were cultured in purely vasculogenic medium for a period of time prior to the addition of osteogenic cues. Physiologically relevant concentrations of exogenous PDGF-BB (e.g., 20 ng/mL) substantially enhanced both vascular network stability and osteogenic differentiation. Comparisons with bone morphogenetic protein-2 (BMP-2), another pro-osteogenic and pro-angiogenic growth factor, indicated that the potential to couple the formation of both lineages might be unique to PDGF-BB. The resulting tissue structure demonstrated the close association of mineral deposits with pre-existing vascular structures that have been described for developing tissues. Accordingly, the presently disclosed combination of a single cell source with a potent induction factor used at physiological concentrations can provide a clinically relevant approach to engineering highly vascularized bone grafts.

The presently disclosed approach can be used to regenerate new bone tissue in large, non-healing segmental defects. This approach depends on the combination of the following three components, which work synergistically to generate functional vascularized bone tissues: (1) stromal vascular fraction (SVF) cells; (2) fibronectin functionalized fibrin hydrogels containing platelet-derived growth factor (PDGF); and (3) three-dimensional printed poly-ε-caprolactone (PCL) scaffolds.

Further, the presently disclosed subject matter provides one or more of the following features: (a) providing stromal vascular fraction (SVF) of cells enriched with autologous microvascular cells; (b) seeding the cells in a hydrogel comprising physiological concentrations of PDGF, which simultaneously enhances both the vascular and osteogenic differentiation of SVF cells to increase tissue survival and integration; (c) modifying thrombin to enable versatility in handling of the hydrogel prior to infusing into scaffolds comprising a biodegradable polymeric material, such as poly-ε-caprolactone (PCL); (d) creating multiple pore geometries to optimize tissue development and integration with host tissues; and (e) modifying the biodegradable polymeric material, e.g., PCL, to increase its oxygen-carrying capacities to enhance survival of cells implanted into ischemic regions and/or to enhance its osteoinductive properties.

A. Scaffold for Treating a Bone Defect

In some embodiments, the presently disclosed subject matter provides a scaffold for regenerating bone tissue, the scaffold comprising a plurality of biodegradable fibers configured to form a porous, three-dimensional (3D) network of fibers, wherein the porous, three-dimensional network of fibers further comprise a hydrogel, and wherein the hydrogel comprises one or more cells encapsulated therein and one or more growth factors capable of promoting regeneration of bone tissue.

In other embodiments, the presently disclosed subject matter provides a biodegradable scaffold for regenerating bone tissue, the scaffold configured to form a porous, three-dimensional (3D) network of interconnected void spaces, wherein the porous, three-dimensional network further comprises a hydrogel, and wherein the hydrogel comprises one or more cells encapsulated therein and one or more growth factors capable of promoting regeneration of bone tissue.

In some embodiments, the scaffold comprises a biodegradable polymer selected from the group consisting of poly-ε-caprolactone (PCL), poly-lactic acid (PLA), poly-glycolic acid (PGA), and poly-lactic-co-glycolic acid (PLGA). In other embodiments, the biodegradable polymer comprises poly-ε-caprolactone (PCL).

As used herein, a “scaffold” is a device or portion thereof that facilitates the ingrowth of tissue within or upon the surface of the device or portion thereof. A scaffold can be a temporary device, which may be substantially or entirely resorbed by the body of a patient having a scaffold implanted therein. In such embodiments, tissue eventually takes up at least a portion of physical space originally occupied by the scaffold.

As used herein, “porosity” means the ratio of the volume of interstices of a material to a volume of a mass of the material.

The term “porous scaffold” refers to a structural matrix, which includes a solid region and an open porous region comprising spaces or discontinuities between adjacent areas of the solid region. The open porous region may be filled with air or gas, at least initially, or it may be at least partially or completely filled with a hydrogel.

As used herein, “osteoinduction” involves the stimulation of osteoprogenitor cells to differentiate into osteoblasts that then begin new bone formation.

Hydrogels are hydrophilic polymer networks, which when filled with water, exhibit swelling. Depending on the composition of the hydrogel polymer network, the swelling can be triggered by a variety of stimuli, including pH, ionic strength, thermal, electrical, ultrasound, and enzyme activities.

The hydrogel can comprise a natural polymer, including, but not limited to, fibrinogen, alginate, gelatin, collagen, hyaluronic acid (HA), chitosan, chondroitin sulfate, dextran sulfate, heparin, heparan sulfate, matrigel, laminin, and the like, and functionalized derivatives thereof, or mixtures of synthetic and natural polymers, for example chitosan-poly(ethylene oxide). Synthetic polymers useful in hydrogel compositions include, but are not limited to, poly(lactide-co-glycolide), polyvinyl alcohol, poly(N-isopropylacrylamide); poly(methacrylic acid-g-polyethylene glycol); polyacrylic acid and poly(oxypropylene-co-oxyethylene)glycol. Accordingly, in some embodiments, the hydrogel comprises a natural polymer selected from the group consisting of fibrinogen, alginate, gelatin, collagen, hyaluronic acid (HA), chitosan, chondroitin sulfate, dextran sulfate, heparin, heparan sulfate, matrigel, and laminin. In other embodiments, the hydrogel comprises fibrinogen.

The polymer(s) comprising the hydrogel may be crosslinked reversibly or irreversibly to form gels adaptable for forming three dimensional tissues (see, e.g., U.S. Pat. Nos. 6,451,346; 6,410,645; 6,432,440; 6,395,299; 6,361,797; 6,333,194; 6,297,337; each of which is incorporated by reference in their entirety.

The hydrogel can include one or more active agents in addition to the living cells and growth factors described herein. According to some embodiments, one or more active agents may be coated on, or otherwise incorporated into, the scaffold. By way of non-limiting example, such “active agents” may include an antibiotic, pain management medication, and/or other biologically active substances. Thus, a biocompatible and biodegradable scaffold, when implanted into a cavity or extraction wound, not only fills the cavity, but also permits the controlled release of biologically active substances. For example, the substance within the pores may be selected such that bacterial growth is hindered, bone formation is accelerated, and/or pain at the bone wound is reduced.

The scaffold in its many embodiments can comprise a resorbable material. A “resorbable” material may include, for example, a biocompatible, bioabsorbable, or biodegradable polymer or other material that is designed to be resorbed by the body of a patient, and eventually replaced with healthy tissue.

As used herein, “biocompatible” refers to the ability of an object to be accepted by and to function in a body of a patient without eliciting a significant foreign body response (such as, for example, an immune, inflammatory, thrombogenic, or like response), and/or are not clinically contraindicated for administration into a tissue or organ.

As used herein, the term “biodegradable” refers to absorbability or degradation of a compound or composition when administered in vivo or under in vitro conditions. Biodegradation may occur through the action of biological agents, either directly or indirectly.

As used herein, “polymer” means a chemical compound or mixture of compounds formed by polymerization and including repeating structural units. Polymers may be constructed in multiple forms and compositions or combinations of compositions. The term “polymer” also is intended to cover copolymer materials and polymer blends formed from two or more polymers. Examples of suitable polymers that may be used include, but are not limited to, biocompatible and/or bioabsorbable polymers or copolymers and combinations thereof. Non-limiting examples biocompatible and/or bioabsorbable polymers or copolymers include, but are not limited to, poly(hydroxy acids), poly(phosphazenes), poly(amino acid-carbonates), poly(anhydrides), and poly(urethanes).

More particularly, biodegradable polymers suitable for use with the presently disclosed scaffolds include synthetic polymers, including, but not limited to, polylactide (PLA), polyglycolide (PGA), poly(lactide-co-glycolide) (PLGA), poly-ε-caprolactone, polydioxanone trimethylene carbonate, polyhybroxyalkonates (e.g., poly(hydroxybutyrate)), poly(ethyl glutamate), poly(DTH iminocarbony(bisphenol A iminocarbonate), poly(ortho ester), and polycyanoacrylates. In particular embodiments, the biodegradable polymer is poly-ε-caprolactone.

In other embodiments, the biodegradable polymer can include a naturally occurring polymer, including, but not limited to, fibrin, casein, serum albumin, collagen, gelatin, lecithin, chitosan, alginate, or poly-amino acids, such as poly-lysine.

One of ordinary skill in the art will appreciate that the selection of a suitable polymer or copolymer depends on several factors. For example, factors in the selection of the appropriate polymer(s) that is used may include bioabsorption (or biodegradation) kinetics; in vivo mechanical performance; cell response to the material in terms of cell attachment, proliferation, migration and differentiation; and biocompatibility. Other relevant factors, which to some extent dictate the in vitro and in vivo behavior of the polymer, include the chemical composition, spatial distribution of the constituents, the molecular weight of the polymer, and the degree of crystallinity.

In some embodiments, the one or more growth factors is selected from the group consisting of a platelet derived growth factor (PDGF), a transforming growth factor-β (TGF-β), a bone morphogenetic protein (BMP), a vascularendothelial cell growth factor (VEGF), and an epithelial cell growth factor (EGF). In particular embodiments, the one or more growth factors comprises a platelet derived growth factor (PDGF).

In some embodiments, the hydrogel comprises one or more living cells. In particular embodiments, the one or more living cells comprise a stromal vascular fraction (SVF) of cells collected from adipose tissue. In other embodiments, one or more cells comprise a stromal vascular fraction (SVF) comprising adipose-derived stromal/stem cells (ASCs) and endothelial cells.

As used herein, the “stromal vascular fraction (SVF)” refers to a heterogeneous mixture of cells isolated by enzymatic dissociation of adipose tissue followed by gradient centrifugation to form a pellet of SVF cells. The pellet of SVF cells contains multipotent mesenchymal cells, which are referred to as adipose-derived stromal/stem cells (ASCs), and endothelial cells.

Stromal vascular fraction (SVF) cells suitable for use with the presently disclosed methods and devices can be isolated from lipoaspirate of a patient in need of treatment for a bone defect using a good manufacturing practice (GMP) device. The isolated SVF cells can then be encapsulated in hydrogel, infused into a scaffold, and then implanted into the defect site. In other embodiments, SVF cells enriched with vascular progenitors also can be used. Autologous cells derived from bone marrow also are suitable for use with the presently disclosed subject matter. Accordingly, in some embodiments, one or more cells comprise autologous cells from bone marrow or adipose tissue.

In yet other embodiments, the hydrogel can comprise so-called “bone-forming cells,” which are those cells suitable for the induction of new bone formation and include those cell types suitable for differentiating into bone cells or suitable for forming a matrix similar to osteoid of forming new bone. Suitable cell types include, but are not limited to stem cells, fibroblast cells, periosteal cells, chondrocytes, osteocytes, pre-osteoblasts, and osteoblasts. Preferably, the stem cells are multipotent, the fibroblast cells are undifferentiated, the periosteal cells are partially differentiated, and the chondrocytes or osteocytes are differentiated. In the case of differentiating cell types, such as fibroblasts or stem cells, these cell types may be placed in close proximity to the ground demineralized bone, which, in the bioreactor and under appropriate conditions, will cause the cells to differentiate into bone cells. In the case of cell types suitable for forming an osteoid-like matrix, such as osteoblasts or chondroblasts, such cell types may be placed in close proximity to the ground demineralized bone in the bioreactor and under appropriate conditions, will cause the cells to synthesize matrix similar to osteoid of forming new bone. The type of cells selected for in vitro bone growth is dependent upon the desired time frame for new bone formation, seeding cell densities, and nutrient medium provided.

The source of the bone-forming cells may be autogenic, allogenic, or xenogenic. In particular embodiments, the cells are autologous. As used herein, the term “autologous” refers to a cell derived or transferred from the same individual, i.e., the donor and recipient of the cell is the same individual. The use of a potential recipient's own cells in the formation of the bone or bone-like biomaterial will result in a tissue unlikely to be rejected for some immunological reason, rendering the transplantable newly formed bone autogenous in nature. The use of allogenic cells in the formation of new bone with subsequent implantation can be achieved by decellularizing any newly formed bone or bone-like structure prior to implantation using any decellularizing technology known in the art depending on the desired characteristics of the acellular bone or bone-like structure desired for a given clinical application.

Three dimensional printed PCL scaffolds can be prepared using a device capable of printing PCL as thin fibers that can be stacked to form 3D scaffolds with intrinsic porosity. The diameter, pore size, pore geometry, and porosity of the fibers within these scaffolds can be varied. Accordingly, the printing device can be used to generate scaffolds of various gross geometries. Such geometries can include anatomical shapes that, in some embodiments, may be highly irregular. In other embodiments, the scaffold is configured to have an anatomical shape. In still other embodiments, the anatomical shape is the shape of a mandible or a maxilla. The information regarding the anatomical shapes can be extracted from CT scans (or any other medical imaging modality that provides 3D shapes of anatomical parts) and fed to the printer.

The PCL also can be modified with one or more vessels capable of delivering oxygen to the cells so that the cells embedded within the scaffolds can receive adequate oxygenation prior to the formation of blood supply. In other embodiments, the biodegradable scaffold further comprises one or more vessels capable of delivering oxygen. In addition, the biodegradable scaffold can further comprise one or more factors capable of inducing osteogenic differentiation of stem cells.

Accordingly, in some embodiments, the scaffold is formed by using three dimensional (3D)-printing. As used herein, the term “three dimensional printing” or 3D printing” refers to a process for making a physical object from a three-dimensional digital model, typically by laying down many successive thin layers of a material. In other embodiments, the scaffold has about a 40% infill density.

B. Method for Preparing a Composite for Treating a Bone Defect

As used herein, the term “composite” is a material, including a device or portion thereof, made of a plurality of components. For example, a “composite” could include a device comprising a scaffold infused with a hydrogel, wherein the hydrogel is seeded with one or more living cells and further comprises one or more growth factors, or the composite can be the hydrogel itself.

Accordingly, in some embodiments, the presently disclosed subject matter provides a method for preparing a composite for promoting regeneration of bone tissue, the method comprising: (a) providing a hydrogel; (b) encapsulating one or more cells in the hydrogel; (c) culturing the encapsulated cells for a first period of time in a vascular medium (VM); (d) waiting for a second period of time, then culturing the encapsulated cells in an osteogenic medium (OM); and (e) adding one or more growth factors to the hydrogel during at least one period of time while the encapsulated cells are cultured in either the VM or the OM.

In some embodiments, the hydrogel comprises a natural polymer selected from the group consisting of fibrinogen, alginate, gelatin, collagen, hyaluronic acid (HA), chitosan, chondroitin sulfate, dextran sulfate, heparin, heparan sulfate, matrigel, and laminin. In particular embodiments, the hydrogel comprises fibrinogen.

In some embodiments, the method further comprises contacting the fibrinogen with thrombin to form a fibrin hydrogel.

In further embodiments, the fibrin hydrogels can be functionalized, in some embodiments, with fibronectin fragments. In yet other embodiments, the fibrin hydrogels can be functionalized with full-length fibronectin. Fibronectin (FN) has specific integrin- and growth factor-(GF) binding motifs. Specifically, the 9th-10th Type III (FN III9-10) repeats provide integrin binding sites, while the 12th-14th Type III (FN III12-14) repeats within the fibronectin protein bind promiscuously to a number of growth factors. These peptide fragments can be cross-linked to fibrin through an a2-plasminogen (a2PI1-8) sequence added recombinantly at their N-terminals. Co-localization of these two fragments to form FN III9-10/12-14 has been demonstrated to work synergistically to aid bone regeneration in a cranial defect.

Bone regeneration can be enhanced by utilizing fibrin modified with FN III9-10/12-14 to encapsulate SVF with physiological concentrations of matrix-bound PDGF, which plays a unique role in inducing SVF cells' self-assembly into vascularized bone. This cell-seeded hydrogel can then be infused into the PCL scaffolds. Plasmids for the FN fragments (FNIII9-10, FNIII12-14, and FN9-10/12-14) can be expressed using bacterial cultures. For example, to incorporate into fibrin, the hydrogels can be prepared to a final concentration of 4 mg/mL fibrin and combined with 2 U/mL thrombin, 4 U/mL factor XIIIa, and 5 mM CaCl₂. An aliquot, e.g., 2 μM, of FN III9-10/12-14 fragment can be cross-linked to fibrinogen through the a2PI1-8 sequence at their N-terminals. The thrombin used to cleave the fibrinogen into fibrin can be modified to be maintained in an inactive state until an ultra-violet (UV) light is shined on it. This modification provides significant versatility to surgeons, who will not be confined, for example, to a 30-second interval (following the addition of thrombin) to seed the hydrogel into the scaffolds.

In some embodiments, the one or more cells comprise adipose-derived stromal/stem cells (ASCs). In particular embodiments, the one or more cells comprise a stromal vascular fraction (SVF) of adipose-derived stromal/stem cells (ASCs). In more particular embodiments, the ASCs are substantially positive for mesenchymal markers CD73 and CD105 and/or substantially negative for markers of the endothelial lineage, CD34+; VEGFR+; CD31+, and the pericyte marker, αSMA. In other embodiments, the ASCs are substantially negative for markers of the endothelial lineage, CD34+; VEGFR+; CD31+ and the endothelial cells are positive for markers of the endothelial linage.

In particular embodiments, the one or more cells are encapsulated in the hydrogel as mono-dispersed cells or as spheroid aggregates, for example, using the hanging drop method as described herein. In other embodiments, the one or more cells are aggregated by culture in non-adherent flasks.

In some embodiments, the one or more growth factors is selected from the group consisting of a platelet derived growth factor (PDGF), a transforming growth factor-β (TGF-β), a bone morphogenetic protein (BMP), a vascular endothelial cell growth factor (VEGF), and an epithelial cell growth factor (EGF). In particular embodiments, the one or more growth factors comprises a platelet derived growth factor (PDGF). In other embodiments, the PDGF is present at a physiological concentration from about 0 ng/mL to about 20 ng/mL. In still other embodiments, the PDGF is present at a supraphysiological concentration from about 20 ng/mL to about 1000 ng/mL. In yet more particular embodiments, the platelet derived growth factor (PDGF) comprises PDGF-BB. Such growth factors can promote regeneration of bone tissue by enhancing vascular stability and osteogenic differentiation of the one or more cells. As used herein, the term “vascular stability” means stabilization of the vessels or ducts that convey fluids.

In some embodiments, the vascular medium (VM) comprises one or more components selected from the group consisting of Endothelial Basal Medium-2, FBS, penicillin/streptomycin, VEGF165, FGF-2, and 1 μg/mL L-ascorbic acid-2-phosphate.

In some embodiments, the osteogenic medium (OM) comprises one or more components selected from the group consisting of low glucose DMEM, FBS, and penicillin/streptomycin, and β-glycerophosphate and L-ascorbic acid-2-phosphate. In yet other embodiments, the osteogenic medium (OM) further comprise one or more components selected from the group consisting of VEGF165, dexamethasone, and FGF-2.

In some embodiments, the method further comprises adding one or more pro-inflammatory cytokines to the hydrogel. In particular embodiments, the one or more pro-inflammatory cytokines comprises IL-1β or tumor necrosis factor alpha (TNFα).

In some embodiments, the method further comprises infusing the composite into a porous, three-dimensional biodegradable scaffold comprising a three-dimensional network of interconnected void spaces.

C. Method for Treating a Bone Defect

In some embodiments, the presently disclosed subject matter provides a method for treating a bone defect, the method comprising contacting the bone defect with a scaffold for regenerating bone tissue, the scaffold as described herein and comprising a plurality of biodegradable fibers configured to form a porous, three-dimensional (3D) network of fibers, wherein the porous, three-dimensional network of fibers further comprise a hydrogel, and wherein the hydrogel comprises one or more cells encapsulated therein and one or more growth factors capable of promoting regeneration of bone tissue.

In some embodiments, the presently disclosed subject matter provides a method for treating a bone defect, the method comprising contacting the bone defect with a biodegradable scaffold for regenerating bone tissue, the scaffold configured to form a porous, three-dimensional (3D) network of interconnected void spaces, wherein the porous, three-dimensional network of further comprise a hydrogel, and wherein the hydrogel comprises one or more cells encapsulated therein and one or more growth factors capable of promoting regeneration of bone tissue.

In some embodiments, the bone defect comprises a critical-sized, non-healing bone defect. In more particular embodiments, the bone defect comprises a bone loss arising from an event selected from the group consisting of a traumatic injury, a disease, surgery, natural aging, radiation, and a congenital defect.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. It will be appreciated that, although not precluded, treating a disease, disorder or condition does not require that the disease, disorder, condition or symptoms associated therewith be completely eliminated.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

The subject treated by the presently disclosed methods in their many embodiments is desirably a human subject, although it is to be understood that the methods described herein are effective with respect to all vertebrate species, which are intended to be included in the term “subject.”

A “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner to make compounds of the disclosure by other methods.

### Example 1

### Materials and Methods

### ASC Isolation

Cellular isolation was performed at the Stem Cell Biology Laboratory, Pennington Biomedical Research Center, under an Institutional Review Board approved protocol (#PBRC 23040) according to published methods. Dubois S. G., et al., Methods Mol Biol 449, 69-79, 2008. Briefly, fresh human subcutaneous adipose lipoaspirate was obtained under informed consent from Caucasian female donors (n=2) undergoing elective liposuction surgery, with an average age of 46 years and average body mass index of 29.1. The lipoaspirate tissue was processed to isolate the adherent population from the stromal vascular cell fraction, as previously described. Hutton D. L., et al., Tissue Eng Part A 18, 1729-40, 2012. The adherent population (“passage 0”) was trypsinized and cryopreserved, Goh B. C., et al., J Tissue Eng Regen Med 1, 322-4, 2007, for shipment to Johns Hopkins University.

### ASC Expansion and Characterization

ASCs were thawed and expanded for one passage in growth medium: high glucose DMEM (GIBCO Invitrogen) with 10% fetal bovine serum (FBS; Atlanta Biologicals), 1% penicillin/streptomycin (GIBCO Invitrogen), and 1 ng/mL FGF-2 (PeproTech). Cells were then trypsinized and used at passage two for all experiments. The phenotypic profile of the cells at this passage was examined via flow cytometry for mesenchymal (CD73, CD105) and vascular markers (CD31, CD34, VEGFR-2, alpha-smooth muscle actin (αSMA)). Briefly, detached cells were suspended in PBS containing 2% FBS and incubated with monoclonal antibodies conjugated to fluorescein isothiocyanate or phycoerythrin for 30 min at 4° C. Cells were analyzed with a flow cytometer (BD Accuri C6). All antibodies were purchased from BD Biosciences.

### Spheroid Formation via Hanging Drop

Cells were trypsinized and resuspended at a concentration of 400,000 cells/mL (this concentration varied in the aggregation study where different cluster sizes were being compared) in culture medium containing 0.24% (w/v) methylcellulose (Sigma). The cell suspension was pipetted as 10 μL drops onto inverted Petri dish caps, which were then reverted and placed on dish bottoms containing sterile water to reduce evaporation. Dishes were incubated at 37° C. overnight to allow cellular aggregation at the air-liquid interface. The dish caps were then flooded with PBS to allow bulk collection and transfer of the spheroids to conical tubes.

### Fibrin Encapsulation

Settled spheroid pellets were resuspended in fibrinogen (8 mg/mL final; Sigma), followed by the addition of thrombin (2.5 U/mL final; Sigma) to initiate gelation. Fibrin gels (35 μL) containing 40 spheroids each (in the aggregation study the number of spheroids per gel was varied to keep the total cell number constant) were pipetted into 6-mm diameter wells and incubated at 37° C. for 30 minutes to allow complete gelation prior to the addition of culture medium. Samples were then cultured for two to three weeks, depending on the experiment, with media changed every other day.

### Media Preparation

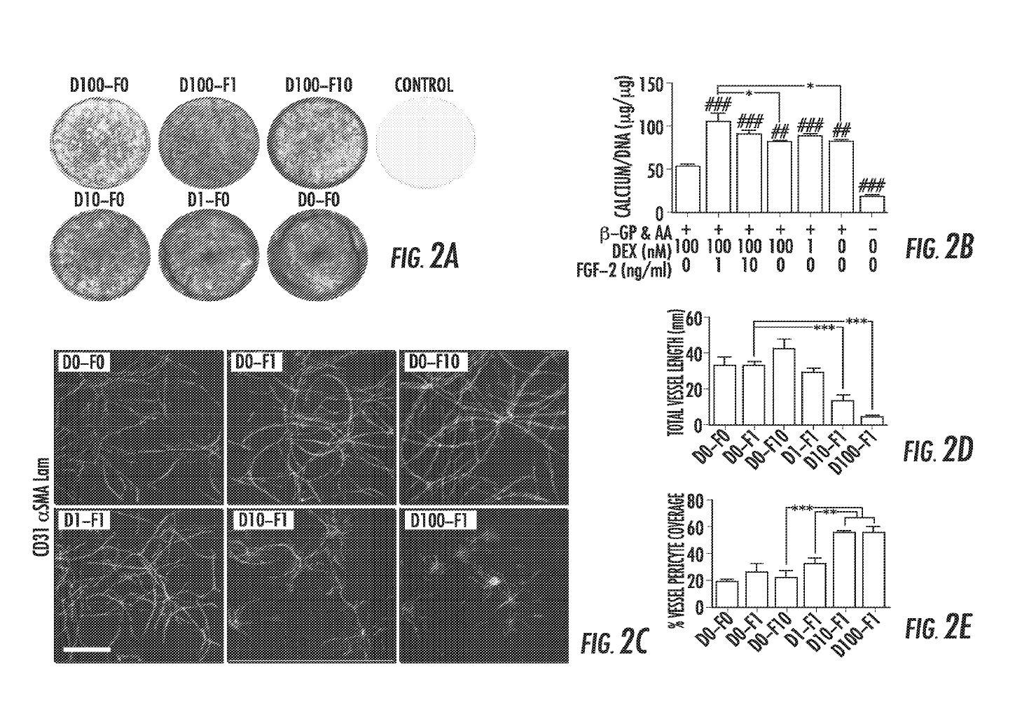

Vascular medium (VM) consisted of: Endothelial Basal Medium-2 (Lonza), 6% FBS, 1% penicillin/streptomycin, 10 ng/mL VEGF165, 1 ng/mL FGF-2, and 1 μg/mL L-ascorbic acid-2-phosphate (Sigma). For monolayer osteogenic differentiation experiments, control medium consisted of low glucose DMEM (GIBCO Invitrogen), 6% FBS, and 1% penicillin/streptomycin; Osteogenic Medium (OM) consisted of control medium plus 10 mM β-glycerophosphate (Sigma) and 50 μM L-ascorbic acid-2-phosphate. For osteogenic differentiation in fibrin gels, OM also included 10 ng/mL VEGF165 and 1 ng/mL FGF-2 to support vascular viability. The composition of FGF-2 and dexamethasone in VM and OM were optimized to support both vascular and osteogenic development (FIG. 2). Media were supplemented with additional growth factors (i.e., PDGF-BB or bone morphogenetic protein-2 (BMP-2)) depending on the experiment. All growth factors were purchased from PeproTech.

### Osteogenic Differentiation Assays

For monolayer experiments, ASCs were seeded at 5000 cells/cm² and cultured in either control medium or OM for 21 days, with the addition of either PDGF-BB or PDGF receptor inhibitor tyrphostin AG1295 (Santa Cruz Biotechnology). Mineralization was assessed via Alizarin Red S staining and quantification of calcium content. For fibrin gel experiments, samples were cultured in VM for up to 8 days followed by up to 12 days of OM supplemented with VEGF and FGF-2. Mineralization was assessed via quantification of calcium content normalized to DNA content. For Alizarin Red S staining, monolayers were washed twice with PBS, fixed with 3.7% formaldehyde for 20 min, and then washed again three times. Samples were subsequently incubated for 10 min with 40 mM Alizarin Red S (Sigma) and washed extensively prior to imaging. To quantify calcium content in each sample, monolayers or whole fibrin gels were washed twice with PBS and then incubated with 0.5 N HCl overnight at 4° C. with agitation. Calcium content in the sample supernatants was quantified using a colorimetric Calcium LiquiColor Test (Stanbio). DNA content was quantified using a PicoGreen dsDNA quantitation kit (Molecular Probes) as previously described. Hutton D. L., et al., Tissue Eng Part A 18, 1729-40, 2012.

### Whole-Mount Immunostaining

All incubation and washing steps were carried out at 4° C. with gentle agitation. Samples were fixed with 3.7% formaldehyde for 3 hrs, and then washed with PBS three times for 30 min each. Fixed gels were carefully removed from their wells and transferred to microcentrifuge tubes for subsequent staining procedures. Gels were permeabilized and blocked for 4 hrs with 0.2% triton X-100 and 5% normal goat serum (Sigma) in PBS with 0.1% Tween (PTw). Samples were then incubated overnight with primary antibodies diluted in blocking solution, followed by three 1-hr washes in PTw. Primary antibodies included: mouse anti-CD31 (4 μg/mL; Santa Cruz Biotechnology) and either rabbit anti-laminin (7 μg/mL; Sigma), rabbit anti-osteocalcin (10 μg/mL; Santa Cruz Biotechnology), or rabbit anti-NG2 (10 μg/mL; Millipore). Samples were then incubated overnight with DyLight 488-conjugated goat anti-mouse and DyLight 649-conjugated goat anti-rabbit (both 1:400; Jackson ImmunoResearch) diluted in blocking solution, followed by three 1-hr washes in PTw. Lastly, samples were blocked in 5% normal mouse serum for 4 hrs, incubated overnight with Cy3-conjugated mouse anti-αSMA (7 μg/mL; Sigma), and then washed three times for 1 hr each. Gels were mounted in 70% glycerol in chamber slides and imaged using a Zeiss LSM 510 confocal microscope with a 5× objective.

### Image Analysis

Confocal z-stacks of immunostained gels were z-projected and thresholded for subsequent quantification of vessel network parameters (n=6 images per group). Thresholded images were analyzed with AngioQuant software, Niemisto A., et al., IEEE Trans Med Imaging 24, 549-53, 2005, to quantify total vessel length, area, and interconnectivity. ImageJ software (NIH) was used to quantify pericyte coverage of vessels, which was defined as αSMA+ area within at least 5 μm of the abluminal face of vessel networks. Briefly, vessel networks were selected in the CD31 channel of thresholded image composites, and selections were enlarged by 5 μm at all edges. These enlarged selections were applied to the αSMA channel, and percent αSMA+ area within the selection was quantified (denoted as “% vessel pericyte coverage”). The mean intensity of the αSMA+ population was quantified by measuring the mean pixel intensity within the non-thresholded αSMA channel viewfield. Lastly, to quantify the amount of basement membrane protein deposited along the vessels, the enlarged vessel selection was applied to the non-thresholded laminin channel, and the mean pixel intensity within the selection area was quantified (denoted as “laminin mean intensity”).

### Statistical Analysis

Quantitative data are expressed as mean±standard error. Multi-group comparisons were determined by one-way ANOVA with Tukey's test for post-hoc analysis. Significance levels are denoted as *P<0.05, **P<0.01, and ***P<0.001.

### Example 2

### Results

### Cell Characterization

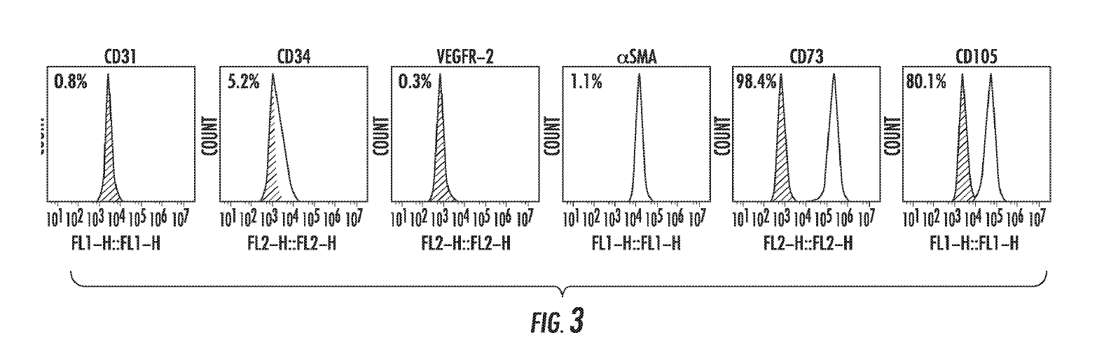

ASCs at passage 2 were primarily positive for mesenchymal markers CD73 (98.4%) and CD105 (80.1%) and predominantly negative for markers of the endothelial lineage (5.2% CD34+; 0.3% VEGFR+; 0.8% CD31+) or the pericyte marker αSMA (1.1%) (FIG. 3).

### Effects of Cellular Aggregation on Vascular Morphogenesis

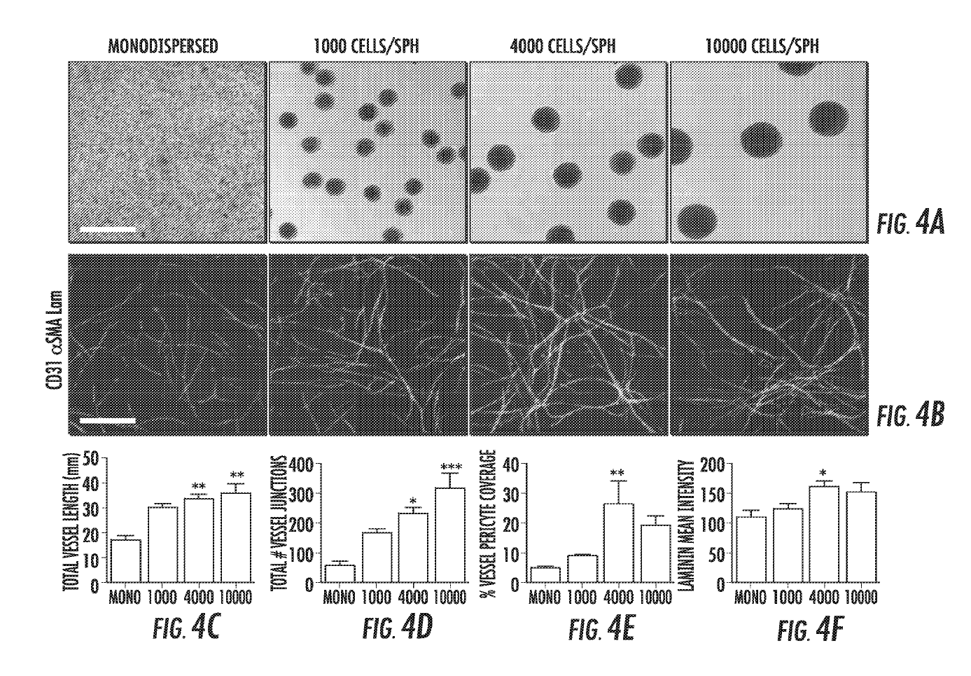

To investigate the effects of cellular aggregation on vascular morphogenesis of ASCs in 3D fibrin gel, cells were encapsulated either as a monodispersed cell suspension or spheroids containing various cell numbers (1,000, 4,000, or 10,000 cells per spheroid) (FIG. 4A). Total cell number remained constant among groups (1.6×10⁵ cells/sample). After 14 days of culture in VM, spheroid aggregates yielded significantly larger (up to 2.1-fold) and more interconnected (up to 5.5-fold) vessel networks than monodispersed cells (FIGS. 4B-D). Vessel coverage with pericytes and laminin also increased with increased initial aggregation, up to 4000 cells per spheroid (FIGS. 4E-F). With the largest spheroid size (10,000 cells per spheroid), the spatial distribution of the networks was less uniform with larger distances between aggregates. Based on these results, spheroid aggregates containing 4,000 cells each were used for the remaining studies.

### Sequential Addition of Factors

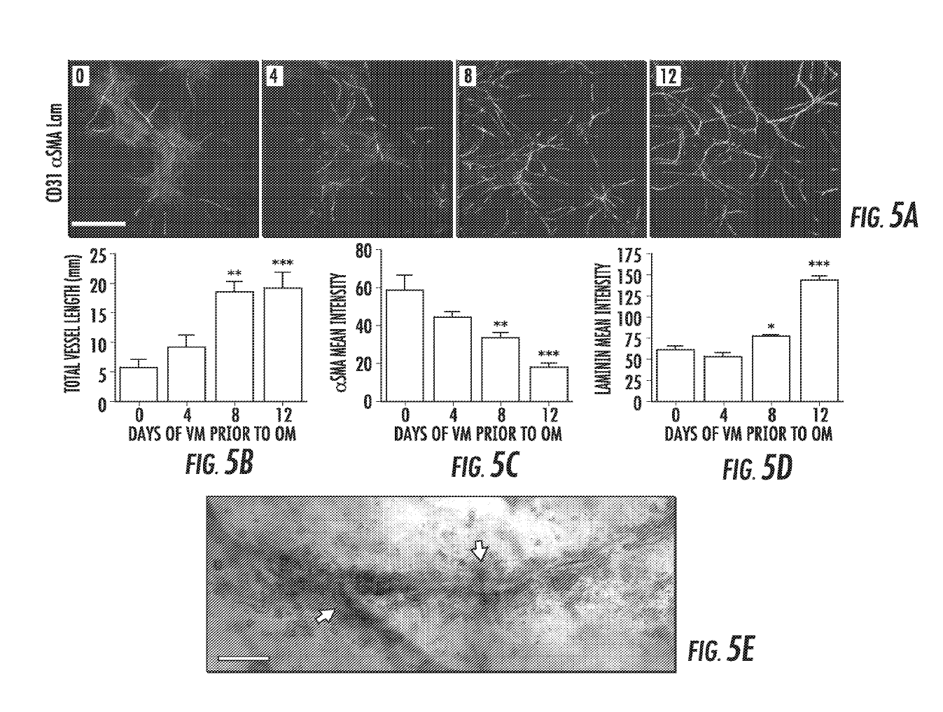

To determine the effects of osteogenic factors on vascular morphogenesis, fibrin-encapsulated spheroids were cultured for 0, 4, 8, or 12 days in VM before being transitioned to OM for the remaining culture period (FIG. 5A). All groups were cultured for a total of 12 days. ASCs cultured in VM for 8 days prior to the addition of osteogenic factors yielded 3.2-fold longer vascular networks than those cultured in OM for the entire period (FIG. 5B). Further, the mean αSMA+ intensity increased significantly with increasing length of time in OM (FIG. 5C), with an apparent increase in the number of αSMA+ and a reduction in their association with the nearby vessels. Extended culture in OM yielded 2.4-fold less laminin deposition around vessels than in VM (FIG. 5D). Thus, while vascular factors were still present in OM, delaying the addition of osteogenic factors by at least 8 days provided the most favorable conditions necessary for vascular network growth. This sequential protocol resulted in early mineral deposits located along vessel tracks (FIG. 5E).

### Independent Effects of Exogenous PDGF-BB on Each Lineage

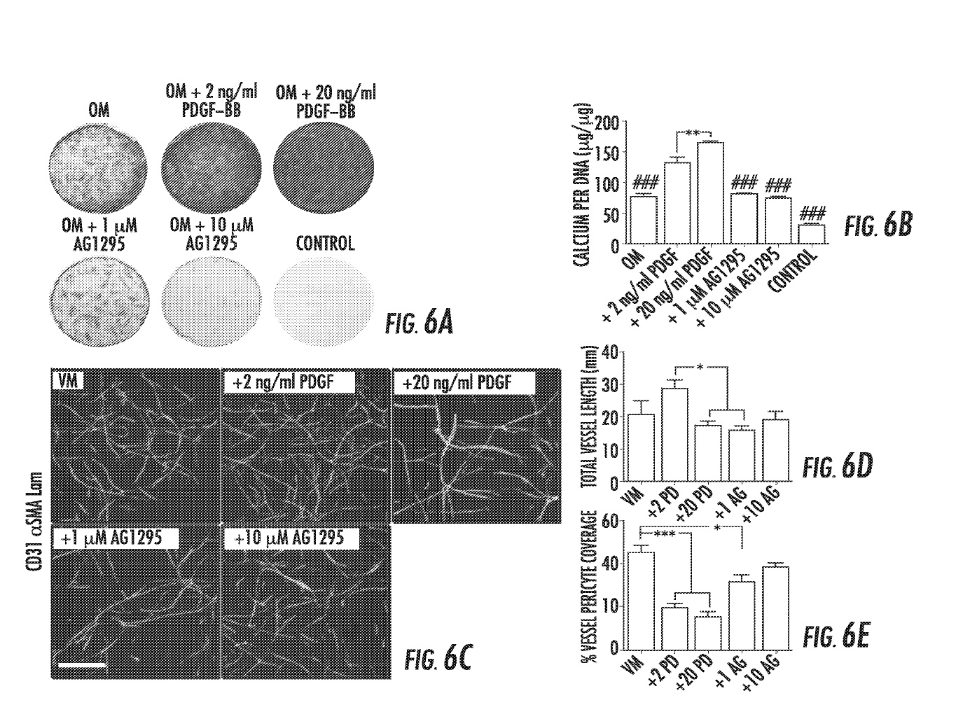

The effects of exogenous PDGF-BB on osteogenic differentiation and vascular morphogenesis of ASCs were studied independently. ASCs were cultured in OM for 21 days with the addition of either PDGF-BB (0 ng/mL, 2 ng/mL, or 20 ng/mL) or PDGF receptor inhibitor AG1295 (0 μM, 1 μM, or 10 μM). In the presence of exogenous PDGF-BB, ASC calcium deposition significantly increased in a dose-dependent manner throughout the culture (FIG. 6A), as well as on a per cell basis (FIG. 6B). In the presence of AG1295, however, total calcium deposition decreased in a dose dependent manner (FIG. 6A), whereas calcium deposition per cell was unchanged relative to the OM only group in the presence of the inhibitor (FIG. 6B).

In a separate example, fibrin-encapsulated spheroids were cultured in VM for 14 days with the addition of PDGF-BB or AG1295. Addition of low concentration (2 ng/mL) PDGF-BB resulted in a slight, 1.4-fold increase in total vessel length (FIGS. 6C-D). Overall, however, PDGF stimulation did not have statistically significant effects on total vessel length (FIG. 6D). Vessels were noticeably wider with the addition of 20 ng/mL PDGF-BB (FIG. 6C). This increased width was reflected quantitatively with statistically larger total vessel area divided by total vessel length (P<0.001, data not shown). Pericyte coverage was reduced by 2.9 fold in the presence of exogenous PDGF-BB, and only slightly reduced with the addition of AG1295 (FIG. 6E).

### Combined Effects of Exogenous PDGF-BB on Vascularized Bone

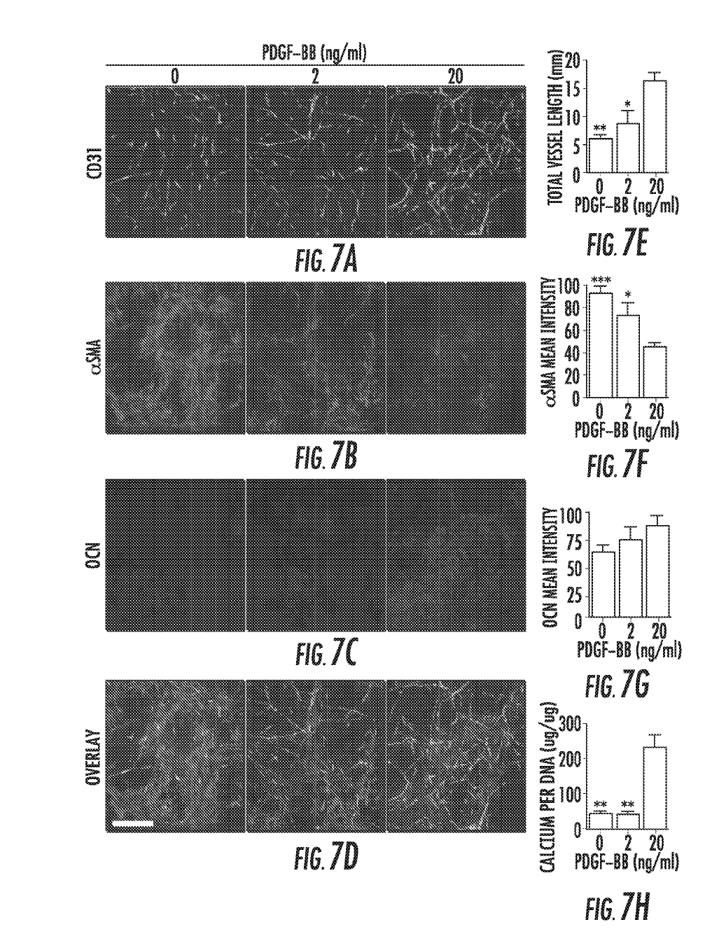

To determine the overall effects of PDGF-BB on the co-development of vasculature and bone within the same culture, encapsulated spheroids were induced in a sequential manner of VM for 8 days followed by OM for 12 days with either 0 ng/mL, 2 ng/mL, or 20 ng/mL PDGF-BB added throughout. Exogenous PDGF-BB resulted in significantly greater vascular density and osteogenic differentiation than cultures without PDGF-BB (FIG. 7D). Specifically, vascular network length was 2.7 fold greater when 20 ng/mL PDGF-BB was added (FIGS. 7A,E), and calcium content per cell was 5.2-fold greater (FIG. 7H). Osteocalcin production was semiquantitatively measured to be 1.4 fold greater, as well (FIGS. 7C,G). Conversely, αSMA intensity was 2.1-fold less in the presence of 20 ng/mL PDGF-BB (FIGS. 7B,F).

In a similar experiment, PDGF-BB was compared with BMP-2 to determine whether these results are unique to PDGF-BB signaling, as BMP-2 also has been shown to be both pro-osteogenic and pro-angiogenic. Encapsulated spheroids were cultured in VM for 8 days followed by OM for 12 days with the addition of either 20 ng/mL PDGF-BB or 20 ng/mL BMP-2. Cultures with PDGF-BB resulted in 4.2 fold greater calcium deposition and 5.6 fold greater calcium per cell than those with BMP-2 (FIG. 7H). Total vascular network length also was 2.3 fold higher in the presence of PDGF-BB than BMP-2 (FIGS. 7A,E).

Transient release of IL-1beta (IL-1β)

Following bone fracture, a transient increase in the expression of pro-inflammatory cytokines occurs at the fracture site. The temporal profile of these cytokines is tightly orchestrated with that of growth factors, such as platelet-derived growth factor (PDGF). The cytokines and the growth factors work to recruit cells and to direct the differentiation pathway of stem cells in the microenvironment. The presently disclosed data demonstrate that IL-1β uniquely stimulates the osteogenic differentiation of adipose-derived stem cells (ASCs), the stem cell population present within the stromal vascular fraction (SVF) of cells isolated from lipoaspirate tissues. The timing of this release is crucial for eliciting the appropriate cellular responses.

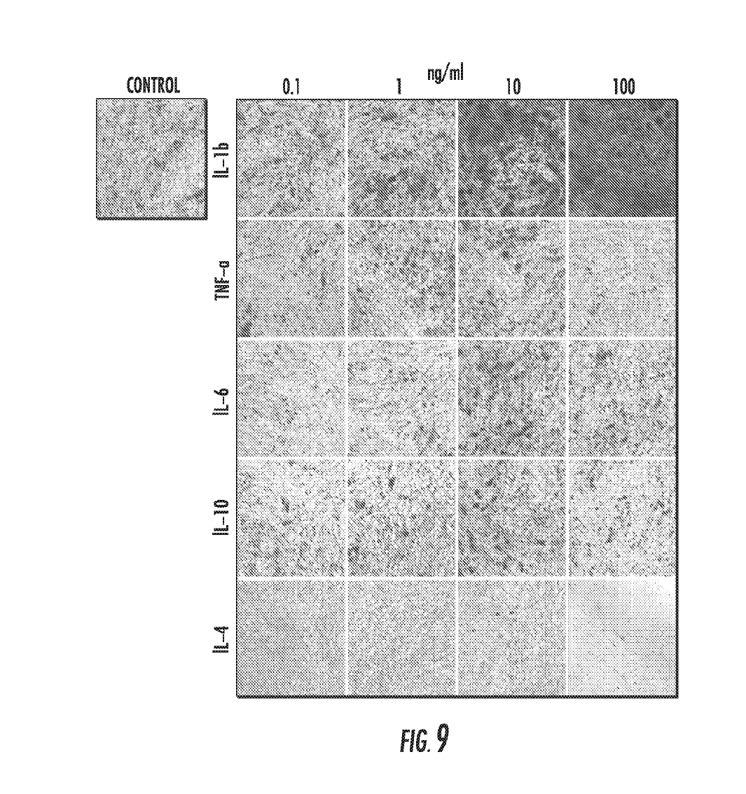

Accordingly, cells were provided with IL-1β only for the first two days and cultured in the absence of IL-1β for the remaining three weeks of culture. A dose dependent effect of IL-1β on ASC differentiation into bone-forming cells was observed. See, for example, FIG. 9, which shows the osteogenic differentiation of ASCs in the absence (Control) or presence of pro- and anti-inflammatory cytokines.

In conjunction with the PDGF-rich hydrogel described hereinabove, IL-1β can be incorporated at these low doses into the hydrogel. The release of this factor can be controlled by diffusion out of the hydrogel since only a burst release is required to elicit the desired biological response.

### Example 3

### Discussion

Engineering vascularized bone has been a challenge in that the factors that are traditionally used in differentiation medium to induce either osteogenic differentiation or angiogenic behaviors can be mutually inhibitory when combined in mixed cultures. Correia C., et al., PLoS One 6, e28352, 2011. The mutual inhibition is particularly devastating for the vascular component, as vessels have proven to be delicate and unstable in the presence of osteogenic cues in vitro. The presently disclosed subject matter demonstrates that exogenous PDGF-BB helps circumvent these issues by enhancing vascular growth and stability, as well as promoting robust osteogenic differentiation and mineralization. Thus, the presently disclosed subject matter demonstrates cooperative growth yielding robust vascular networks within dense mineralized matrix.

Previous studies demonstrated that cultured ASCs contain minute vascular subpopulations that are fully capable of forming stable vascular structures on 2D substrates when seeded at high densities. Hutton D. L., et al., Tissue Eng Part A 18, 1729-40, 2012. One observation in that earlier study was that the cells spontaneously self-assembled into dense clusters of cells prior to extensive vascular growth, suggesting that heterotypic cell-cell interactions were important to the process. Without wishing to be bound to any one particular theory, the presently disclosed subject matter confirms that cellular aggregation facilitates formation of dense, stable vascular networks by ASCs. The aggregation of ASCs into spheroids prior to encapsulation into 3D fibrin gels enhanced their resulting vascular network density and interconnectivity, pericyte coverage, and deposition of vascular basement membrane.

This finding complements previous work demonstrating that aggregation of pure endothelial cells into spheroids or on the surface of microbeads reduces apoptosis and greatly enhances vascular sprouting. Martineau L., Doillon C. J., Angiogenesis 10, 269-77, 2007; Korff T., Augustin H. G., J Cell Biol 143, 1341-52, 1998. ASCs specifically have been shown to reside in perivascular sites in native adipose tissues, Crisan M., et al., Cell Stem Cell 3, 301-13, 2008; Traktuev D. O., et al., Circ Res 102, 77-85, 2008, and stabilize vascular networks in vitro, Traktuev D. O., et al., Circ Res 102, 77-85, 2008; Verseijden F., et al., Tissue Eng Part A 16, 101-14, 2010. Therefore, aggregation also may serve to bring the pericyte-like cells closer to the endothelial sub-population to help support and stabilize them.

While ASC spheroids were able to sprout dense vascular networks in VM, vascular growth was severely limited in OM, which consisted of VM plus β-glycerophosphate and higher ascorbic acid. β-glycerophosphate is an essential initiator of mineralization, as cells cannot synthesize their own phosphate. It may be a possible source of vascular inhibition, however, after it is broken down into inorganic phosphate by alkaline phosphatase activity. Bellows C. G., et al., Bone Miner 17, 15-29, 1992. High inorganic phosphate levels (>2.5 mM) have been shown to induce endothelial cell apoptosis via increased generation of reactive oxygen species. Di Marco G. S., et al., Am J Physiol Renal Physiol 294, F1381-7, 2008.

To compensate for this effect, delaying the addition of osteogenic factors by one week allowed the vessels to grow and establish stable networks prior to inducing osteogenic differentiation of the non-endothelial sub-population. This sequential process is similar to what occurs in bone development and fracture repair in which invading vasculature is a prerequisite for the entry of osteogenic progenitors and subsequent mineralization. Maes C., et al., Dev Cell 19, 329-44, 2010; Mikos A. G., et al., Tissue Eng 12, 3307-39, 2006. This established vascular network serves to bring nutrients and progenitor cells to the highly active site of regeneration, Caplan A. I., Correa D. J Orthop Res 29, 1795-803, 2011; Carano R. A., Filvaroff E. H., Drug Discov Today 8, 980-9, 2003, as well as serving as a template around which mineral will be deposited. Mikos A. G., et al., Tissue Eng 12, 3307-39, 2006. Establishment of vascular networks prior to osteogenesis helped to stabilize the networks with pericytes and increased laminin deposition, possibly shielding them from the inhibitory factors in the second phase.

The presently disclosed subject matter also demonstrates that exogenous PDGF-BB at physiological concentrations, e.g., 20 ng/mL, further helps to circumvent issues of mutual inhibition by enhancing both the osteogenic and vascular development of ASCs. In osteogenic cultures, PDGF-BB markedly increased mineral deposition overall and on a per cell basis. Conversely, addition of the inhibitor AG1295 reduced overall mineral deposition, while the relative amount per cell remained unchanged.

These results suggest that PDGF signaling might not be required for osteogenic differentiation of ASCs, but it may significantly amplify their responsiveness to osteogenic cues, leading to the production of greater amounts of mineral. This response may be somewhat unique to ASCs, as previous studies have shown that PDGF signaling either has no effect or even an inhibitory effect on the osteogenic differentiation bone marrow-derived MSCs. Chaudhary L. R., et al., Bone 34, 402-11, 2004; Kumar A., et al., Tissue Eng Part A 16, 983-93, 2010; Tanaka H., Liang C. T., J Cell Physiol 164, 367-75, 1995; Gruber R., et al., Platelets 15, 29-35, 2004; Tokunaga A., et al., J Bone Miner Res 23, 1519-28, 2008.

In vascular cultures, PDGF signaling had minimal effects on vascular network properties, with only low levels of exogenous PDGF-BB contributing to a slight increase in network density. PDGF signaling did, however, significantly affect pericyte coverage of the vessels. Exogenous PDGF-BB caused a significant decrease in pericyte coverage, likely by masking endogenous gradients and stimulating the pericytes to migrate randomly. Interestingly, the inhibitor AG1295 did not significantly reduce pericyte coverage. This observation is possibly a result of the cellular aggregation prior to encapsulation, in which the growing vessels are already in direct contact with pericyte-like cells, thereby reducing the necessity to recruit pericytes via a PDGF gradient.

PDGF-BB appeared to play a different role in maintaining vascular stability in the presence of osteogenic factors. Using a sequential protocol of a vascular phase followed by an osteogenic phase, the addition of 20 ng/mL PDGF-BB was able to support vascular growth and stability, as well as amplify mineral deposition. Some hypotheses have been proposed in the literature about how these effects occur. For example, a recent review discusses the possible roles of PDGF-BB signaling in terms of bone fracture repair. Caplan A. I., Correa D., J Orthop Res 29, 1795-803, 2011.

When bone is injured, high concentrations of PDGF-BB may cause vascular pericytes and other osteoprogenitors to populate the injury site and increase production of mineral. These cells also may respond to PDGF-BB by secreting higher levels of VEGF to recruit angiogenesis towards the regenerating tissue. Caplan A. I., Correa D., J Orthop Res 29, 1795-803, 2011; Sato N., et al., Am J Pathol 142, 1119-30, 1993. This increased endogenous production of VEGF may be furthering the stability of established vessels in the osteogenic environment. It also may be possible that the vessels in these cultures are responding directly to the exogenous PDGF-BB via PDGF receptors. Beitz J. G., et al., Proc Natl Acad Sci USA 88, 2021-5, 1991; Battegay E. J., et al., J Cell Biol 125, 917-28, 1994.

An observation in one study was that the expression of αSMA changed dramatically throughout the induction period. αSMA aids cellular contractility and is primarily expressed by vascular smooth muscle cells and pericytes. Allt G., Lawrenson J. G., Cells Tissues Organs 169, 1-11, 2001. In that study, initial ASC cultures exhibited only 1% αSMA positive population. Prior to the application of osteogenic supplements, αSMA+ cells were primarily located in perivascular locations. Following exposure to osteogenic supplements, however, a dramatic increase in the intensity and number of cells expressing this marker was observed. This change in expression profile reflects what is observed in native mineralizing bone: Osteoblasts upregulate expression of αSMA during active mineralization, whereas resting osteoblasts and osteocytes do not express αSMA. Kinner B., Spector M., J Orthop Res 20, 622-32, 2002.

The presently disclosed subject matter, however, also showed that ASCs treated with 20 ng/mL PDGF-BB in the presence of osteogenic cues exhibited substantially lower αSMA expression at day 20 than those cultured without PDGF-BB even though the mineral content in PDGF-BB cultures was significantly higher. This observation suggests that a αSMA expression alone does not directly correlate with increased osteogenesis.

Lastly, whether this mutually beneficial response exhibited by cells in the presence of PDGF-BB was unique by comparison to BMP-2, the most widely studied and clinically applied pro-osteogenic growth factor, was examined BMP-2 has been shown to significantly enhance osteogenic differentiation and in vivo bone growth, Schmitt J. M., et al., J Orthop Res 17, 269-78, 1999, as well as indirectly enhance vascular growth via promotion of VEGF production. Deckers M. M., et al., A. Endocrinology 143, 1545-53, 2002.

The presently disclosed subject matter shows through direct comparison that PDGF-BB is considerably more effective than BMP-2 at both amplifying osteogenic differentiation of ASCs and maintaining vascular stability when applied at 20 ng/mL. Importantly, this concentration of PDGF (20 ng/mL) is only 5 to 10 times the concentration in normal human serum, Czarkowska-Paczek B., et al., J Physiol Pharmacol 57, 189-97, 2006, and is within the physiological range measured during bone injury. Giannoudis P. V., et al., Bone 42, 751-7, 2008. Clinical safety is imperative, as supraphysiological concentrations may lead to adverse side effects such as bone overgrowth and inflammation. Carragee E. J., et al., Spine J 11, 471-91, 2011. Together, these results indicate that, in combination with ASCs, PDGF-BB may be better suited than BMP-2 for the enhancement of both bone and vasculature at physiological concentrations.

In summary, the presently disclosed subject matter demonstrates that a single population of ASCs can be driven to form robust vascularized bone with the addition of exogenous PDGF-BB. Cellular aggregation was important in encouraging heterotypic cell-cell interactions and inducing dense, stable vascular networks. These factors, together with a delayed addition of osteogenic cues were essential to recapitulating intrinsic developmental profiles that instructed the cells to grow into complex tissue grafts with minimal manipulation compared to existing models. This induction protocol using physiological levels of PDGF-BB can be utilized to engineer vascularized bone grafts with greater efficiency and potential for subsequent integration and functionality.

### Example 4

### Engineering Anatomically Shaped Vascularized Bone Grafts with hASCs and 3D-Printed PCL Scaffolds

### Introduction

Treatment of large craniomaxillofacial (CMF) bone defects due to trauma or resection presents unique challenges due to the complex, three-dimensional (3D) geometry of the bone (Harris and Laughlin, 2013; Gentile et al., 2013). Currently, there remains no satisfactory solution for critical-sized, geometrically complex CMF defects. The current gold standard of treatment, the autologous bone graft, is limited in its ability to aesthetically reproduce the patient's facial features and requires an additional, often painful surgery to harvest bone for the graft. The amount of bone harvested is limited, and complications at the harvesting site such as pain, infection, and bleeding can lead to additional donor-site morbidity (Brydone et al., 2010). Tissue-engineered bone grafts are rapidly becoming promising alternatives, allowing precise tailoring of the graft's shape and eliminating the need for additional surgeries and co-morbidities (Reichert et al., 2012; Li, Hsu et al., 2011; Li, Zhang et al., 2011; Cesarano et al., 2005; Lee et al., 2009; Klammert et al., 2010; Wang et al., 2009).

Both synthetic and native scaffolds have been used for bone regeneration. Each of these has their relative advantages and disadvantages. Synthetic biomaterial scaffolds may not precisely mimic the structural or biochemical cues provided by native scaffolds, such as decellularized bone grafts (Grayson et al., 2010). However, the fabrication process facilitates much greater control over the material characteristics and reproducibility of the grafts. Specifically, their material properties, shape, bioactivity, and porosity can be closely controlled and customized for specific applications. Polycaprolactone (PCL) has emerged as a favorable polymer for scaffold fabrication, as it is biocompatible and safely breaks down in the body at a rate similar to new bone formation (Lam et al., 2009; Woodruff and Hutmacher, 2010). Furthermore, the polymer has already received regulatory approval for certain applications (Woodruff and Hutmacher, 2010). PCL is also highly amenable for use in additive manufacturing (AM) technologies. AM is commonly termed ‘3D printing’ and is a particularly promising technology for scaffold fabrication. With extrusion-based 3D printing, a thermoplastic biomaterial, such as PCL, can be melted and extruded in a computer-controlled pattern to construct scaffolds layer-by-layer (Wei et al., 2012; Park et al., 2012; Hamid et al., 2011; Shim et al., 2012) and can be effectively leveraged to design patient-specific, customized parts. In addition, 3D printing can be used to regulate the internal architecture of the scaffold and its gross geometry. In prior applications, 3D models of the desired bone have been extracted from patient computed tomography (CT) scans (Ono et al., 1992; Sun and Lal, 2002; Ballyns et al., 2008), providing a blueprint for a personalized scaffold that interfaces with the defect site and recreates the appropriate anatomical features.

In this Example, a method is presented for 3D printing anatomically shaped PCL scaffolds with varying internal pore structures using a custom-designed 3D printer. The suitability of these scaffolds was assessed for seeding cellular aggregates. Specifically, human adipose-derived stem cells (hASCs) were used, an easily accessible cell source with huge clinical potential. Previous efforts have demonstrated that ASCs can give rise to vascular and osteogenic lineages cooperatively (Correia et al., forthcoming; Guven et al., 2011; Hutton et al., 2013). In this study, the scaffold porosity was determined that facilitates uniform cell seeding and subsequent vascularized bone formation. The potential to engineer porous, 3D-printed PCL scaffolds with these appropriate porosities in the shape of human mandibular and maxillary bones was demonstrated.

### Materials and Methods

3D Printing of PCL Scaffolds:

A Syil X4 CNC mill (Syil America, Coos Bay, Oreg.) was converted into a 3D printer by attaching a custom hot-melt pressure extruder to the spindle of the mill. An Ultimus V (Nordson EFD, Providence, R.I.) regulator controlled the extruder pressure and a nozzle heater maintained the melt temperature at a set value. The printer was run at a linear speed of 2.7 mm/s (determined through optimization studies). PCL (Capa 6400; Perstorp, Perstorp, Sweden) was used in pellet form for printing. The printer dispensed PCL through a 460 μm diameter nozzle onto a heated bed. The temperature of the bed was maintained at roughly 40° C. to ensure that the bottom layer of PCL remained attached to the print surface and did not warp as the print cooled. A 120 mm fan was used to cool the scaffold during printing. For the optimization, characterization, and cell seeding studies, cuboidal scaffolds (15×15×5 mm) were generated as CAD models, exported as stereolithography (STL) files, and imported into Slic3r, an open-source program used to generate machine G-code. The infill density was varied from 20 to 80% to generate scaffolds with varying pore sizes. As used herein, the term “infill density” refers to the material used to fill the empty space inside the shell of an object. Infill is generally measured by percentage. To evaluate the quality of the printed scaffolds (output) relative to the CAD code (input), a cross-correlation image analysis script was developed. The script compared top-down stereomicroscope images of the scaffold to a theoretical input by superimposing the two images to obtain a quantitative correlation factor, a measurement of the accuracy of the scaffold's pore size and shape relative to the theoretical ideal. 40% infill density scaffolds were printed at temperatures between 70 and 120° C. and the correlation factor was calculated for each.

Scanning Electron Microscopy:

Pores were also analyzed using scanning electron microscopy (SEM). Samples were sputter-coated with platinum and imaged at 25 and 55× magnifications on a JEOL 6700F microscope (JEOL USA, Peabody, Mass.). The 25× magnification images were used to measure the pore size. The widths of five random pores per image were measured and the mean and standard deviation calculated. This data was used to plot a relationship between measured pore size and infill density.

hASCs Isolation and Aggregate Formation:

The hASCs were isolated from fresh subcutaneous lipoaspirate tissue that was obtained from Caucasian female donors with informed consent, according to a Johns Hopkins Institutional Review Board approved protocol. Cellular isolation was performed as previously described (Dubois et al., 2008). In brief, tissue was digested with 1 mg/mL type I collagenase (Worthington Biochemical Co., Lakewook, N.J.) for 1 h at 37° C., centrifuged to obtain the stromal vascular fraction (SVF) pellet, then plated onto tissue culture plastic to obtain the plastic-adherent population (passage 0 hASCs). The hASCs were expanded in growth medium: high-glucose DMEM (Gibco Invitrogen), 10% fetal bovine serum (FBS; Atlanta Biologicals), 1% penicillin/streptomycin (P/S; Gibco, Grand Island, N.Y.), and 1 ng/mL fibroblast growth factor-2 (FGF-2; PeproTech, Rocky Hill, N.J.). Cells were used at passage two for all experiments. The hASCs were aggregated through suspension culture. In brief, monodispersed cells were suspended in growth medium containing 0.24% (w/v) methylcellulose, then dispensed into petri dishes coated with 2% (w/v) agarose to minimize cellular adherence to the dish. After overnight incubation in suspension, cellular aggregates were collected with a pipet, and then centrifuged before encapsulation procedures. Aggregates ranged in size from 50-300 nm.

Scaffold Preparation and Cell Seeding:

Square PCL sheets were printed for cell seeding experiments, and cylindrical hollow punches were used to obtain scaffolds of the appropriate size. For the cell seeding distribution study, scaffolds with infill densities ranging from 20 to 80% were prepared with dimensions 8 mm (d)×5 mm (h). For all other cell-based studies, scaffolds with 40% infill density were prepared with dimensions 4 mm (d)×2 mm (h). Scaffolds were then treated with 3.0M sodium hydroxide for 1 h to increase surface hydrophilicity, sterilized with 70% ethanol for 1 h, rinsed with PBS, and then incubated with growth medium at 37° C. for at least 1 h.

For cell seeding, ASC aggregates were collected and resuspended at 3×10⁷ cells/mL in a mixture of fibrinogen (8 mg/mL; Sigma) and thrombin (2 U/mL; Sigma). Cylindrical scaffolds were blotted dry on sterile Kim wipes to remove medium from pores. The cell suspension was pipetted unto one end of the scaffolds to fill the pore spaces. The fibrin was allowed to fully polymerize at 37° C. for 30 min before the addition of culture medium. To assess cell seeding distribution, scaffolds were fixed with 3.7% formaldehyde, cut in half to expose the center, and then stained with 4′,6-diamidino-2-phenylindole (DAPI).

Vascular and Osteogenic Induction:

For vascular induction, scaffolds were cultured in vascular medium (VM) for 14 days with medium changed every 2 days. VM consisted of endothelial basal medium-2 (Lonza, Walkersville, Md.), 6% FBS, 1% P/S, 10 ng/mL vascular endothelial growth factor (rh-VEGF-165, PeproTech, Rocky Hill, N.J.), 1 ng/mL FGF-2, and 1 μg/mL L-ascorbic acid-2-phosphate (Sigma, St. Louis, Mo.). Scaffolds were then either implanted for in vivo studies or fixed with 3.7% formaldehyde for assessment through whole-mount immunostaining.

For osteogenic induction, scaffolds were cultured in osteogenic medium (OM) for 14 days with medium changed every 2-3 days. OM consisted of low-glucose DMEM (Gibco, Grand Island, N.Y.), 6% FBS, 1% P/S, 10 mM β-glycerophosphate (Sigma, St. Louis, Mo.), and 50 μM L-ascorbic acid-2-phosphate. Scaffolds were subsequently fixed with 3.7% formaldehyde and assessed through histology.

In Vivo Implantation of Scaffold Constructs:

All animal procedures were conducted according to a protocol approved by the Johns Hopkins University Institutional Animal Care and Use Committee and NIH guidelines for the care and use of laboratory animals (NIH Publication #85-23 Rev. 1985) were observed. Sterile PCL scaffolds (4 mm (d)×2 mm (h)) were seeded with either fibrin only, fibrin+ASC aggregates (‘uncultured’), or fibrin+ASC aggregates followed by 18 days of in vitro vascular induction (‘cultured’). Male athymic nude rats (7 weeks old, n=2 per group; Charles River Laboratories, Frederick, Md.) were anesthetized with isoflurane. Small lateral incisions were made in the dorsal region of the skin, into which subcutaneous implants were inserted. The skin was sutured closed, and rats were monitored closely to ensure full recovery from anesthesia. All rats were sacrificed 7 days post-implantation for retrieval of scaffold implants. Samples were fixed for 48 h in 10% neutral buffered formalin before histological analysis.

Immunostaining and Histological Analysis:

For histological analysis, fixed samples were paraffin-embedded, cut into 5 μm sections, deparaffinized, and then rehydrated for staining. Hematoxylin and eosin (H&E) staining was used to assess general tissue morphology and cellular distribution. Osteogenic samples were stained with von Kossa and van Gieson to assess mineral deposition Immunofluorescence staining was used to assess vascular growth of in vitro cultivated samples through whole-mount staining, and the in vivo explants through paraffin-embedded sections. Whole-mount immunostaining was performed as previously described (Hutton et al., 2013), using the following primary antibodies: mouse anti-CD31 (Sigma, St. Louis, Mo.), mouse anti-alpha-smooth muscle actin (αSMA; Sigma, St. Louis, Mo.), and rabbit anti-NG2 (Santa Cruz Biotech, Santa Cruz, Calif.). Rehydrated paraffin-embedded sections were treated with heat-mediated antigen retrieval in 10 mM citrate buffer before blocking in 10% normal goat serum/0.5% Triton X-100. The primary antibodies used were rabbit anti-CD31 (Abcam, Cambridge, Mass.) and mouse anti-αSMA (Sigma, St. Louis, Mo.). All fluorescently labeled secondary antibodies were purchased from Jackson Immunoresearch (West Grove, Pa.). Fluorescence and brightfield images were obtained with an inverted Zeiss Axio Observer microscope.

Anatomically Shaped Scaffolds:

A computerized tomography (CT) head scan of a child was imported into Mimics (Materialise, Leuven, Belgium) and 3D models of the maxilla and mandible were segmented by hand using the default bone thresholding setting. Models were smoothed and wrapped to fill any large holes, while ensuring that important surface details were not lost. The models were exported as STL files and code was generated in Slic3r (open-source) using an infill density of 40%. Both models were printed with automatically generated support structure. All support structure was printed with the same PCL material. The support structures were trimmed away after the completion of the print.

### Results

Scaffold Characterization:

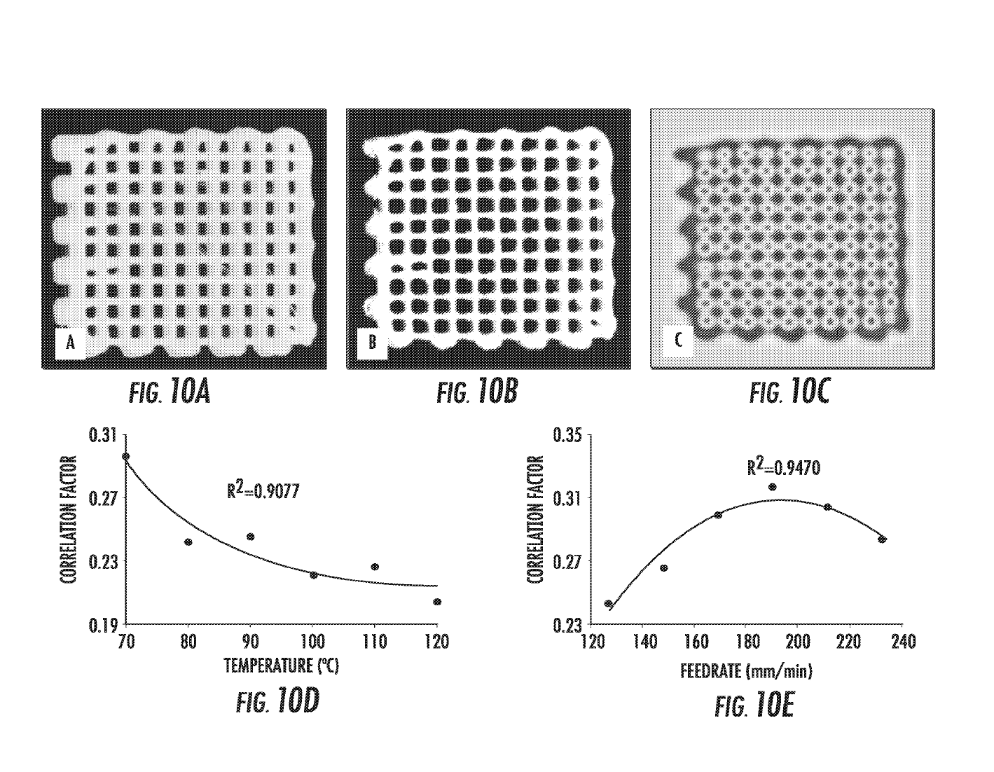

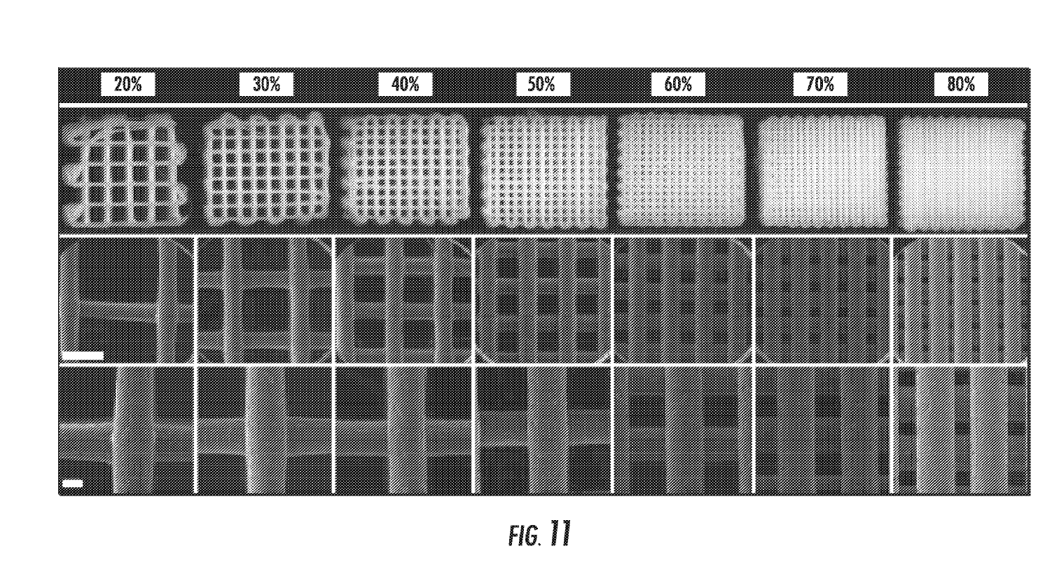

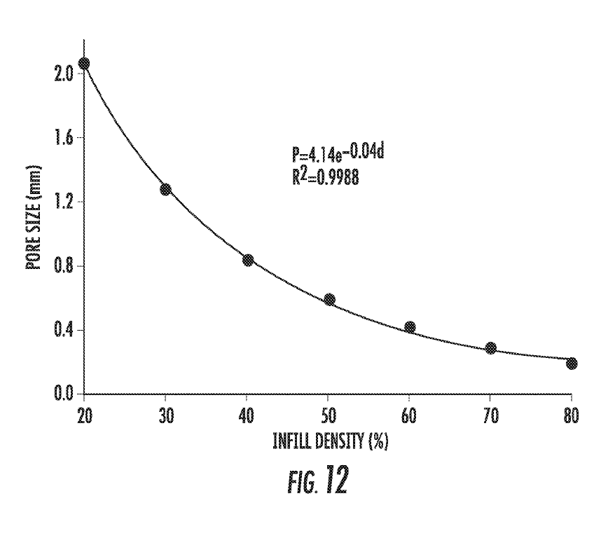

Cross-correlation analysis between scaffolds of varying porosities and the theoretical pore yielded a correlation factor indicative of the relative accuracy and regularity of the scaffold pores (FIG. 10). The correlation factor was highest at the lowest melt temperature, 70° C. Scaffolds at all porosities were rectangular, although higher infill densities yielded more geometrically accurate scaffolds (FIG. 11). At low infill densities, the fiber being printed had fewer attachment points to the layer beneath it, meaning it could be easily be displaced when the printer made rapid movements. This can be observed in the top left corner of the 20% scaffold. Analysis of SEM data shows an exponential relationship between pore size and infill density, approximated by the function P=4.14e^{0.04d}, where P represents measured pore size and d represents infill density (FIG. 12). The standard deviation of these pore size measurements was negligible.

Cell Seeding Distribution:

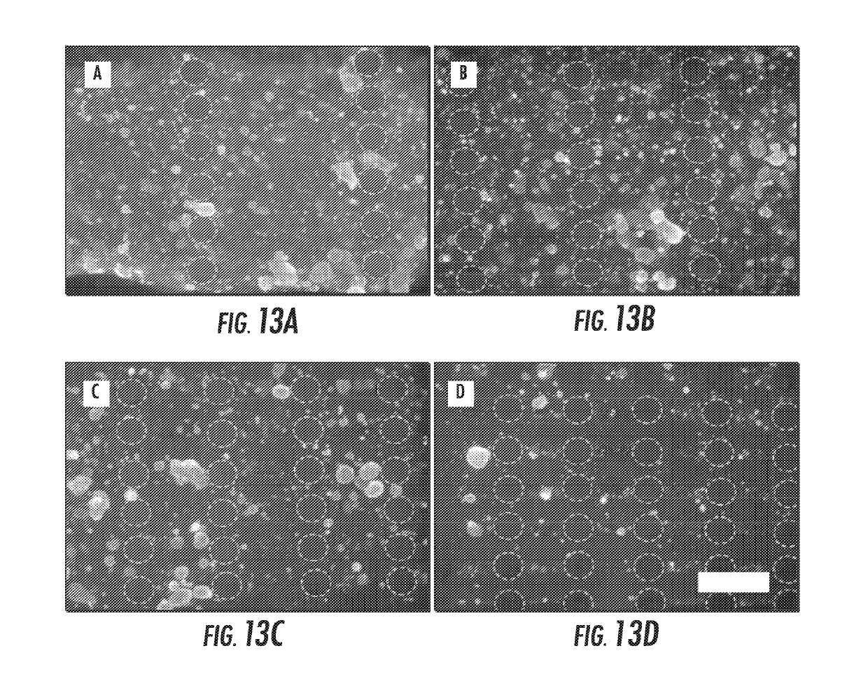

Although cells were seeded into all scaffolds, fluorescent images of DAPI stains show that at lower infill densities (20 and 30%) larger cell-aggregates settled to the bottom of the scaffolds, while at 50% the larger aggregates did not adequately penetrate into the scaffold. The most uniform seeding was achieved using the scaffolds with 40% infill density (FIG. 13).

Vessel Formation and Mineral Deposition:

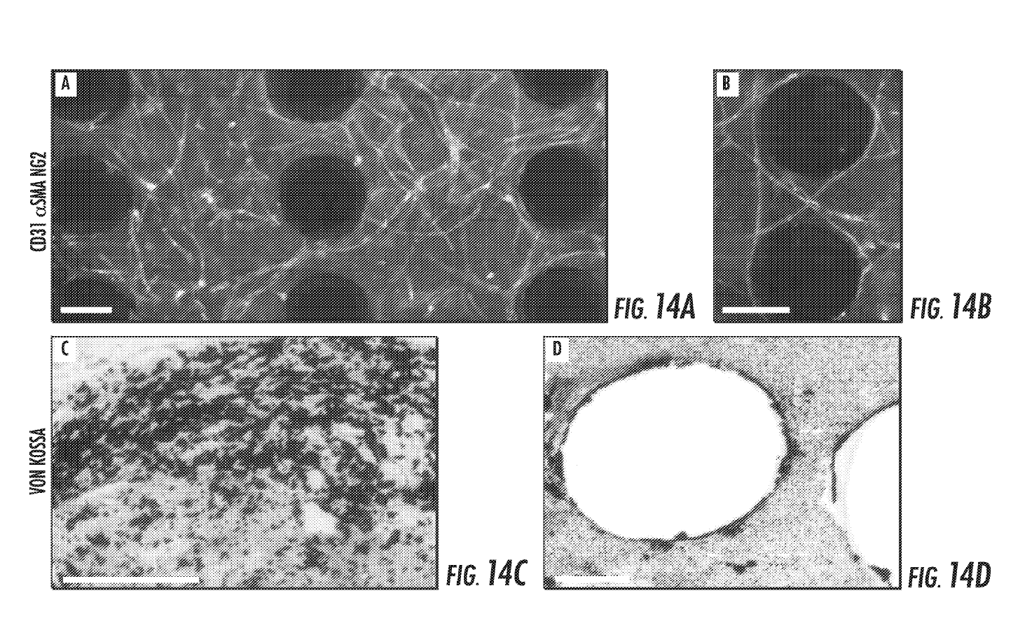

After 14 days of culture in VM, ASCs formed extensive vascular networks throughout the fibrin-filled pore spaces of the scaffold (FIG. 14A). These CD31+ vessels were covered with pericyte-like cells that stained positively for αSMA and/or NG2. Vessels near PCL fibers were often wrapped along the surface of the fiber (FIG. 14B). Scaffolds cultured in OM demonstrated dense mineral deposits within the pore spaces of the scaffold (FIG. 14C), with additional mineral lining the surfaces of PCL fibers (FIG. 14(D).

In Vivo Vascularization:

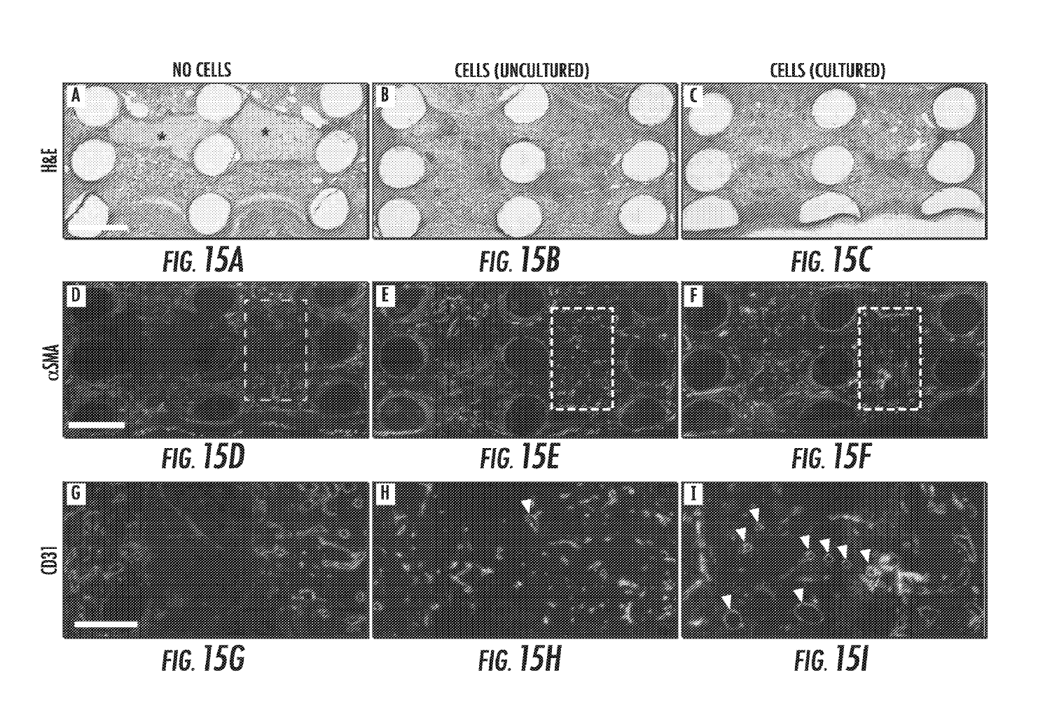

After 7 days in vivo, acellular scaffolds were infiltrated with host cells near the outer regions, while still containing sparsely infiltrated remnants of fibrin within the central region (FIG. 15A). Conversely, cell-seeded scaffolds were densely populated with cells throughout the entire sample (FIG. 15B-15C). Similar observations were made regarding vascular infiltration: acellular scaffolds were only vascularized in the outer regions (FIG. 15D) while cell-seeded scaffolds had a greater density of CD31+ cells throughout the central regions (FIG. 15E-15F). A higher magnification view of the central regions of each scaffold demonstrates that prevascularized scaffolds contained more lumen containing, pericyte stabilized vessels than those that were uncultured or implanted without cells (FIG. 15G-15I).

Anatomically Shaped Scaffolds:

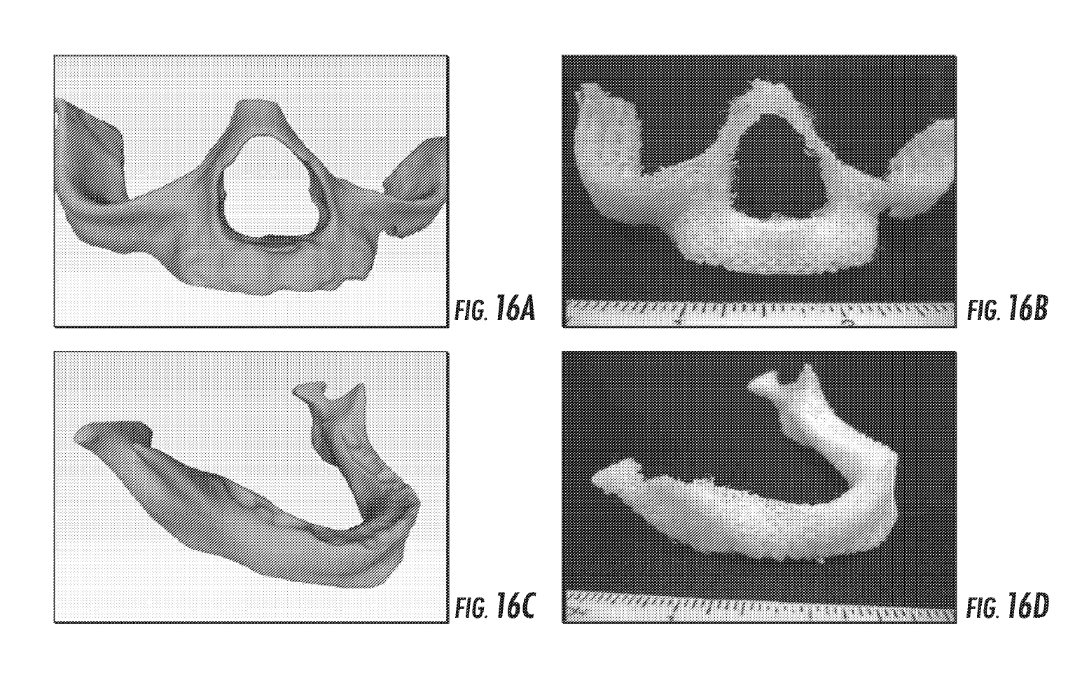

After trimming away the support structures, the anatomically shaped scaffolds closely resembled the 3D models, from which they were printed (FIG. 16). All gross anatomical features were replicated in the scaffolds, with consistent, regular pores maintained throughout.

### Discussion

Prior studies have investigated the potential for engineering anatomically shaped temporomandibular joint (TMJ) condylar bone grafts using native and synthetic materials (Feinberg et al., 2001; Hollister et al., 2000; Alhadlaq et al., 2004; Weng et al., 2001; Alhadlaq and Mao, 2003). It has been shown herein that this concept can be expanded for engineering large bones in the CMF region using 3D printing. This study sought to determine optimal scaffold parameters that facilitate vascularized bone formation by hASCs and to produce clinically sized, anatomically shaped scaffolds with complex geometries. Using a validated cross-correlation image analysis technique to assess the accuracy of the print at different melt temperatures, it was determined that the temperature that provided the best fit for printing was 70° C., closest to the melting point of PCL (60° C.). At that temperature, the fiber strands solidified most quickly. However, printing at the lower temperatures sacrificed printing speed. A printing temperature of 80° C. was ultimately selected for the subsequent studies to ensure sufficient accuracy with faster printing. The negligible standard deviation of pore size measurements demonstrated the capability of the 3D printer to reproducibly generate regular, controlled porosity over a broad range of infill densities.

Human ASCs are a promising, clinically relevant cell source for engineering vascularized bone. Prior studies have typically used co-cultures of bone marrow-derived mesenchymal stem cells and a mature endothelial population (Correia et al., 2011; Rivron et al., 2012; Tsigkou et al., 2010). However, previous work recently demonstrated that hASCs can be readily directed to form integrated vascularized bone tissues. The hASCs were formed into aggregates before encapsulation and seeding. Aggregated cells were used based on previous studies that demonstrated significantly improved vascular growth by aggregated hASC cultures compared to monodispersed samples (Hutton et al., 2013). Additional studies have also demonstrated improved in vivo cellular retention and regenerative properties following aggregation (Amos et al., 2010; Cheng et al., 2012). However, the larger, heavier nature of cell aggregates made them more prone to settling due to gravity and/or clogging within pores as compared to monodispersed cells. Therefore, a critical component of this study was to establish a porosity, in which hASC aggregates could be effectively seeded and uniformly distributed throughout the scaffolds to enable subsequent uniform tissue development. The largest cell aggregates were roughly 300 μm, hence the denser scaffolds (i.e., infill density>60%) were not used as their pore sizes were too small to facilitate cellular infiltration. However, the requirement for progressively larger pores was counter-balanced by the need to keep the aggregates suspended in the scaffold. The aggregates were suspended in fibrinogen to deliver into the scaffolds. Consequently, ensuring a quick rate of fibrin coagulation when mixed with thrombin was important for homogenous cell distribution throughout scaffolds with larger pores. Among the infill densities that were tested in this study, it was found that 40% was optimal for uniform cell seeding. At this particular infill density (a pore size of about 800 μm), cell aggregates were dispersed evenly throughout the scaffold pores. For greater pore sizes, aggregates tended to settle to the bottom of the scaffolds and for small pore sizes, large aggregates clogged the pores, preventing uniform dispersion of cells. Next, both vascular and osteogenic differentiation of ASCs seeded in scaffolds were induced with optimized scaffold parameters. The formation of both robust vascular networks and significant mineral deposition were able to be induced in vitro. These tissues formed throughout the fibrin-filled pores. However, there was some evidence that vessels and mineral were more densely wrapped along the surface of PCL fibers (FIG. 14). These differential cellular responses throughout the composite scaffold can be further examined.

In vivo results indicated that after 1 week, hASC-seeded scaffolds contained greater cellularity and increased vascular density within the central regions compared to acellular scaffolds. This suggests that seeding cells within the scaffolds before implantation might provide a benefit with regard to tissue formation within the graft. Prevascularization of hASCs for 18 days before implantation accelerated the formation of these vessels. This has been previously indicated with mature and progenitor endothelial cell populations within gel-based implants (Chen et al., 2009; Singh et al., 2011; Unger et al., 2010). This study is the first to demonstrate more rapid vascularization of a composite scaffold using clinically relevant hASC populations.

Finally, the 3D printing technology was applied to clinically relevant geometries for CMF reconstruction. In particular, the potential to generate porous scaffolds that replicated the incredibly complex anatomies of the mandible and maxilla were explored. These scaffolds faithfully replicated complicated geometric features on each of these bones such as the TMJ condyle and the maxillary opening to the nasal cavity observed in the 3D models. Furthermore, the anatomically shaped scaffolds maintained the same level of porosity observed in the rectangular scaffolds, allowing for cell seeding and vascularization for future studies. The 3D correlation between the 3D models and printed scaffolds can be quantitatively assessed through micro-CT and the potential of anatomically shaped scaffolds for vascularization in vivo, specifically in orthotopic animal models, also can be assessed. In particular, whether bone regenerated using this approach would maintain the appropriate geometry long term in response to physiological cues can be assessed.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Quarto N., Longaker M. T. FGF-2 inhibits osteogenesis in mouse adipose tissue-derived stromal cells and sustains their proliferative and osteogenic potential state. Tissue Eng 12, 1405-18, 2006.
Meury T., Verrier S., Alini M. Human endothelial cells inhibit BMSC differentiation into mature osteoblasts in vitro by interfering with osterix expression. J Cell Biochem 98, 992-1006, 2006.
Hori Y., Hu D. E., Yasui K., Smither R. L., Gresham G. A., Fan T. P. Differential effects of angiostatic steroids and dexamethasone on angiogenesis and cytokine levels in rat sponge implants. Br J Pharmacol 118, 1584-91, 1996.
Correia C., Grayson W., Eton R., Gimble J. M., Sousa R. A., Reis R. L., et al. Human adipose-derived cells can serve as a single-cell source for the in vitro cultivation of vascularized bone grafts. J Tissue Eng Regen Med 2012 doi: 10.1002/term.1564.
Correia C., Grayson W. L., Park M., Hutton D., Zhou B., Guo X. E., et al. In vitro model of vascularized bone: synergizing vascular development and osteogenesis. PLoS One 6, e28352, 2011.
Tsigkou O., Pomerantseva I., Spencer J. A., Redondo P. A., Hart A. R., O'Doherty E., et al. Engineered vascularized bone grafts. Proc Natl Acad Sci USA 107, 3311-6, 2010.
Rouwkema J., de Boer J., Van Blitterswijk C. A. Endothelial cells assemble into a 3-dimensional prevascular network in a bone tissue engineering construct. Tissue Eng 12, 2685-93, 2006.
Liu Y., Teoh S. H., Chong M. S., Lee E. S., Mattar C. N., Randhawa N. K., et al. Vasculogenic and osteogenesis-enhancing potential of human umbilical cord blood endothelial colony-forming cells. Stem Cells 30, 1911-24, 2012.
Usami K., Mizuno H., Okada K., Narita Y., Aoki M., Kondo T., et al. Composite implantation of mesenchymal stem cells with endothelial progenitor cells enhances tissue engineered bone formation. J Biomed Mater Res A 90, 730-41, 2009.
Koob S., Torio-Padron N., Stark G. B., Hannig C., Stankovic Z., Finkenzeller G. Bone formation and neovascularization mediated by mesenchymal stem cells and endothelial cells in critical-sized calvarial defects. Tissue Eng Part A 17, 311-21, 2011.
Nishi M., Matsumoto R., Dong J., Uemura T. Engineered bone tissue associated with vascularization utilizing a rotating wall vessel bioreactor. J Biomed Mater Res A 101(2):421-7, 2013.
Gimble J., Guilak F. Adipose-derived adult stem cells: isolation, characterization, and differentiation potential. Cytotherapy 5, 362-9, 2003.
Zuk P. A., Zhu M., Ashjian P., De Ugarte D. A., Huang J. I., Mizuno H., et al. Human adipose tissue is a source of multipotent stem cells. Mol Biol Cell 13, 4279-95, 2002.
Cao Y., Sun Z., Liao L., Meng Y., Han Q., Zhao R. C. Human adipose tissue-derived stem cells differentiate into endothelial cells in vitro and improve postnatal neovascularization in vivo. Biochem Biophys Res Commun 332, 370-9, 2005.
Zonari A., Novikoff S., Electo N. R., Breyner N. M., Gomes D. A., Martins A., et al. Endothelial differentiation of human stem cells seeded onto electrospun polyhydroxybutyrate/polyhydroxybutyrate-co-hydroxyvalerate fiber mesh. PLoS One 7, e35422, 2012.
Hutton D. L., Logsdon E. A., Moore E. M., Mac Gabhann F., Gimble J. M., Grayson W. L. Vascular morphogenesis of adipose-derived stem cells is mediated by heterotypic cell-cell interactions. Tissue Eng Part A 18, 1729-40, 2012.
Miranville A., Heeschen C., Sengenes C., Curat C. A., Busse R., Bouloumie A. Improvement of postnatal neovascularization by human adipose tissue-derived stem cells. Circulation 110, 349-55, 2004.
Planat-Benard V., Silvestre J. S., Cousin B., Andre M., Nibbelink M., Tamarat R., et al. Plasticity of human adipose lineage cells toward endothelial cells: physiological and therapeutic perspectives. Circulation 109, 656-63, 2004.
Boquest A. C., Noer A., Sorensen A. L., Vekterud K., Collas P. CpG methylation profiles of endothelial cell-specific gene promoter regions in adipose tissue stem cells suggest limited differentiation potential toward the endothelial cell lineage. Stem Cells 25, 852-61, 2007.
Koh Y. J., Koh B. I., Kim H, Joo H. J., Jin H. K., Jeon J., et al. Stromal vascular fraction from adipose tissue forms profound vascular network through the dynamic reassembly of blood endothelial cells. Arterioscler Thromb Vasc Biol 31, 1141-50, 2011.
Gardin C., Bressan E., Ferroni L., Nalesso E., Vindigni V., Stellini E., et al. In vitro concurrent endothelial and osteogenic commitment of adipose-derived stem cells and their genomical analyses through comparative genomic hybridization array: novel strategies to increase the successful engraftment of tissue-engineered bone grafts. Stem Cells Dev 21, 767-77, 2012.
Güven S., Mehrkens A., Saxer F., Schaefer D. J., Martinetti R., Martin I., et al. Engineering of large osteogenic grafts with rapid engraftment capacity using mesenchymal and endothelial progenitors from human adipose tissue. Biomaterials 32, 5801-9, 2011.
Müller A. M., Mehrkens A., Schafer D. J., Jaquiery C., Guven S., Lehmicke M., et al. Towards an intraoperative engineering of osteogenic and vasculogenic grafts from the stromal vascular fraction of human adipose tissue. Eur Cell Mater 19, 127-35, 2010.
Liu Y., Chan J. K., Teoh S. H. Review of vascularised bone tissue-engineering strategies with a focus on co-culture systems. J Tissue Eng Regen Med 2012.
Caplan A. I., Correa D. PDGF in bone formation and regeneration: new insights into a novel mechanism involving MSCs. J Orthop Res 29, 1795-803, 2011.
Armulik A., Abramsson A., Betsholtz C. Endothelial/pericyte interactions. Circ Res 97, 512-23, 2005.
Bergers G., Song S. The role of pericytes in blood-vessel formation and maintenance. Neuro Oncol 7, 452-64, 2005.
Solheim E. Growth factors in bone. Int Orthop 22, 410-6, 1998.
Andrew J. G., Hoyland J. A., Freemont A. J., Marsh D. R. Platelet-derived growth factor expression in normally healing human fractures. Bone 16, 455-60, 1995.
Graham S., Leonidou A., Lester M., Heliotis M., Mantalaris A., Tsiridis E. Investigating the role of PDGF as a potential drug therapy in bone formation and fracture healing. Expert Opin Investig Drugs 18, 1633-54, 2009.
Park Y. J., Lee Y. M., Park S. N., Sheen S. Y., Chung C. P., Lee S. J. Platelet derived growth factor releasing chitosan sponge for periodontal bone regeneration. Biomaterials 21, 153-9, 2000.
Nash T. J., Howlett C. R., Martin C., Steele J., Johnson K. A., Hicklin D. J. Effect of platelet-derived growth factor on tibial osteotomies in rabbits. Bone 15, 203-8, 1994.
Chaudhary L. R., Hofmeister A. M., Hruska K. A. Differential growth factor control of bone formation through osteoprogenitor differentiation. Bone 34, 402-11, 2004.
Kumar A., Salimath B. P., Stark G. B., Finkenzeller G. Platelet-derived growth factor receptor signaling is not involved in osteogenic differentiation of human mesenchymal stem cells. Tissue Eng Part A 16, 983-93, 2010.
Tanaka H., Liang C. T. Effect of platelet-derived growth factor on DNA synthesis and gene expression in bone marrow stromal cells derived from adult and old rats. J Cell Physiol 164, 367-75, 1995.
Gruber R., Karreth F., Kandler B., Fuerst G., Rot A., Fischer M. B., et al. Plateletreleased supernatants increase migration and proliferation, and decrease osteogenic differentiation of bone marrow-derived mesenchymal progenitor cells under in vitro conditions. Platelets 15, 29-35, 2004.
Tokunaga A., Oya T., Ishii Y., Motomura H., Nakamura C., Ishizawa S., et al. PDGF receptor beta is a potent regulator of mesenchymal stromal cell function. J Bone Miner Res 23, 1519-28, 2008.
Lee J., Gupta D., Panetta N.J., Levi B., James A. W., Wan D., et al. Elucidating mechanisms of osteogenesis in human adipose-derived stromal cells via microarray analysis. J Craniofac Surg 21, 1136-41, 2010.
Dubois S. G., Floyd E. Z., Zvonic S., Kilroy G., Wu X., Carling S., et al. Isolation of human adipose-derived stem cells from biopsies and liposuction specimens. Methods Mol Biol 449, 69-79, 2008.
Goh B. C., Thirumala S., Kilroy G., Devireddy R. V., Gimble J. M. Cryopreservation characteristics of adipose-derived stem cells: maintenance of differentiation potential and viability. J Tissue Eng Regen Med 1, 322-4, 2007.
Niemisto A., Dunmire V., Yli-Harja O., Zhang W., Shmulevich I. Robust quantification of in vitro angiogenesis through image analysis. IEEE Trans Med Imaging 24, 549-53, 2005.
Pittenger M. F., Mackay A. M., Beck S. C., Jaiswal R. K., Douglas R., Mosca J. D., et al. Multilineage potential of adult human mesenchymal stem cells. Science 284, 143-7, 1999.
Martineau L., Doillon C. J. Angiogenic response of endothelial cells seeded dispersed versus on beads in fibrin gels. Angiogenesis 10, 269-77, 2007.
Korff T., Augustin H. G. Integration of endothelial cells in multicellular spheroids prevents apoptosis and induces differentiation. J Cell Biol 143, 1341-52, 1998.
Crisan M., Yap S., Casteilla L., Chen C. W., Corselli M., Park T. S., et al. A perivascular origin for mesenchymal stem cells in multiple human organs. Cell Stem Cell 3, 301-13, 2008.
Traktuev D. O., Merfeld-Clauss S., Li J., Kolonin M., Arap W., Pasqualini R., et al. A population of multipotent CD34-positive adipose stromal cells share pericyte and mesenchymal surface markers, reside in a periendothelial location, and stabilize endothelial networks. Circ Res 102, 77-85, 2008.
Verseijden F., Posthumus-van Sluijs S. J., Pavljasevic P., Hofer S. O., van Osch G. J., Farrell E. Adult human bone marrow- and adipose tissue-derived stromal cells support the formation of prevascular-like structures from endothelial cells in vitro. Tissue Eng Part A 16, 101-14, 2010.
Bellows C. G., Heersche J. N., Aubin J. E. Inorganic phosphate added exogenously or released from beta-glycerophosphate initiates mineralization of osteoid nodules in vitro. Bone Miner 17, 15-29, 1992.
Di Marco G. S., Hausberg M., Hillebrand U., Rustemeyer P., Wittkowski W., Lang D., et al. Increased inorganic phosphate induces human endothelial cell apoptosis in vitro. Am J Physiol Renal Physiol 294, F1381-7, 2008.
Maes C., Kobayashi T., Selig M. K., Torrekens S., Roth S. I., Mackem S., et al. Osteoblast precursors, but not mature osteoblasts, move into developing and fractured bones along with invading blood vessels. Dev Cell 19, 329-44, 2010.
Mikos A. G., Herring S. W., Ochareon P., Elisseeff J., Lu H. H., Kandel R., et al. Engineering complex tissues. Tissue Eng 12, 3307-39, 2006.
Carano R. A., Filvaroff E. H. Angiogenesis and bone repair. Drug Discov Today 8, 980-9, 2003.
Sato N., Beitz J. G., Kato J., Yamamoto M., Clark J. W., Calabresi P., et al. Plateletderived growth factor indirectly stimulates angiogenesis in vitro. Am J Pathol 142, 1119-30, 1993.
Beitz J. G., Kim I S, Calabresi P., Frackelton A. R., Jr. Human microvascular endothelial cells express receptors for platelet-derived growth factor. Proc Natl Acad Sci USA 88, 2021-5, 1991.
Battegay E. J., Rupp J., Iruela-Arispe L., Sage E. H., Pech M. PDGF-BB modulates endothelial proliferation and angiogenesis in vitro via PDGF beta-receptors. J Cell Biol 125, 917-28, 1994.
Allt G., Lawrenson J. G. Pericytes: cell biology and pathology. Cells Tissues Organs 169, 1-11, 2001.
Kinner B., Spector M. Expression of smooth muscle actin in osteoblasts in human bone. J Orthop Res 20, 622-32, 2002.
Schmitt J. M., Hwang K., Winn S. R., Hollinger J. O. Bone morphogenetic proteins: an update on basic biology and clinical relevance. J Orthop Res 17, 269-78, 1999.
Deckers M. M., van Bezooijen R. L., van der Horst G., Hoogendam J., van Der Bent C., Papapoulos S. E., et al. Bone morphogenetic proteins stimulate angiogenesis through osteoblast-derived vascular endothelial growth factor A. Endocrinology 143, 1545-53, 2002.
Czarkowska-Paczek B., Bartlomiejczyk I., Przybylski J. The serum levels of growth factors: PDGF, TGF-beta and VEGF are increased after strenuous physical exercise. J Physiol Pharmacol 57, 189-97, 2006.
Giannoudis P. V., Pountos I., Morley J., Perry S., Tarkin H. I., Pape H. C. Growth factor release following femoral nailing. Bone 42, 751-7, 2008.
Carragee E. J., Hurwitz E. L., Weiner B. K. A critical review of recombinant human bone morphogenetic protein-2 trials in spinal surgery: emerging safety concerns and lessons learned. Spine J 11, 471-91, 2011.
Simmons, C. A., Alsberg, E., Hsiong, S., Kim, W. J., & Mooney, D. J. (2004). Dual growth factor delivery and controlled scaffold degradation enhance in vivo bone formation by transplanted bone marrow stromal cells. Bone, 35(2), 562-569.
Griffith, L. G., and Naughton, G. (2002). Tissue engineering-current challenges and expanding opportunities. Science, 295(5557), 1009-1014.
Harris C M, Laughlin R. Reconstruction of hard and soft tissue maxillofacial defects. Atlas Oral Maxillofac Surg Clin North Am 2013; 21:127-138.
Gentile M A, Tellington A J, Burke W J, Jaskolka M S. Management of midface maxillofacial trauma. Atlas Oral Maxillofac Surg Clin North Am. 2013; 21:69-95.
Brydone A S, Meek D, Maclaine S. Bone grafting, #orthopaedic|biomaterials, and the clinical need for bone engineering. Proc Inst Mech Eng H 2010; 224:1329-1343.
Reichert J C, Cipitria A, Epari D R, Saifzadeh S, Krishnakanth P, Berner A, Woodruff M A, Schell H, Mehta M, Schuetz M A, Duda G N, Hutmacher D W. A tissue engineering solution for segmental defect regeneration in load-bearing long bones. Sci Transl Med 2012; 4:141ra93.
Li J H, Hsu Y C, Luo E, Khadka A, Hu J. Computer-aided design and manufacturing and rapid prototyped nanoscale hydroxyapatite/polyamide (n-HA/PA) construction for condylar defect caused by mandibular angle ostectomy. Aesthet Plast Surg 2011; 35:636-640.
Li J H, Zhang L, Lv S Y, Li S J, Wang N, Zhang Z T. Fabrication of individual scaffolds based on a patient-specific alveolar bone defect model. J Biotechnol 2011; 151:87-93.
Cesarano J, Dellinger J G, Saavedra M P, Gill D D, Jamison R D, Grosser B A, Sinn-Hanlon J M, Goldwasser M S. Customization of load-bearing hydroxyapatite lattice scaffolds. Int J Appl Ceram Technol 2005; 2:212-220.
Lee C H, Marion N W, Hollister S, Mao J J. Tissue formation and vascularization in anatomically shaped human joint condyle ectopically in vivo. Tissue Eng A. 2009; 15:3923-3930.
Klammert U, Gbureck U, Vorndran E, Rodiger J, Meyer-Marcotty P, Kubler A C. 3D powder printed calcium phosphate implants for reconstruction of cranial and maxillofacial defects. J Craniomaxillofac Surg 2010; 38:565-570.
Wang P, Hu J, Ma P X. The engineering of patient-specific, anatomically shaped, digits. Biomaterials 2009; 30:2735-2740.
Grayson W L, Fr [symbol] ohlich M, Yeager K, Bhumiratana S, Chan M E, Cannizzaro C, Wan L Q, Liu X S, Guo X E, Vunjak-Novakovic G. Engineering anatomically shaped human bone grafts. Proc Natl Acad Sci USA 2010; 107:3299-3304.
Lam C X F, Hutmacher D W, Schantz J T, Woodruff M A, Teoh S H. Evaluation of polycaprolactone scaffold degradation for 6 months in vitro and in vivo. J Biomed Mater Res A 2009; 90A:906-919.
Woodruff M A, Hutmacher D W. The return of a forgotten polymer-Polycaprolactone in the 21st century. Prog Polym Sci. 2010; 35:1217-1256.
Wei C, Cai L, Sonawane B, Wang S F, Dong J Y. High-precision flexible fabrication of tissue engineering scaffolds using distinct polymers. Biofabrication 2012; 4:025009.
Park S H, Park D S, Shin J W, Kang Y G, Kim H K, Yoon T R, Shin J W. Scaffolds for bone tissue engineering fabricated from two different materials by the rapid prototyping technique: PCL versus PLGA. J Mater Sci Mater Med 2012; 23:2671-2678.
Hamid Q, Snyder J, Wang C, Timmer M, Hammer J, Guceri S, Sun W. Fabrication of three-dimensional scaffolds using precision extrusion deposition with an assisted cooling device. Biofabrication 2011; 3:034109.
Shim J H, Lee J S, Kim J Y, Cho D W. Bioprinting of a mechanically enhanced three-dimensional dual cell-laden construct for osteochondral tissue engineering using a multi-head tissue/organ building system. J Micromech Microeng 2012; 22:085014.
Ono I, Ohura T, Narumi E, Kawashima K, Matsuno I, Nakamura S, Ohhata N, Uchiyama Y, Watanabe Y, Tanaka F, Kishinami T. 3-Dimensional analysis of craniofacial bones using 3-dimensional computer-tomography. J Craniomaxillofac Surg 1992; 20:49-60.
Sun W, Lal P. Recent development on computer aided tissue engineering—A review. Comput Methods Programs Biomed 2002; 67:85-103.
Ballyns J J, Gleghorn J P, Niebrzydowski V, Rawlinson J J, Potter H G, Maher S A, Wright T M, Bonassar L J. Image-guided tissue engineering of anatomically shaped implants via MRI and micro-CT using injection molding. Tissue Eng Part A 2008; 14:1195-202.
Guven S, Mehrkens A, Saxer F, Schaefer D J, Martinetti R, Martin I, Scherberich A. Engineering of large osteogenic grafts with rapid engraftment capacity using mesenchymal and endothelial progenitors from human adipose tissue. Biomaterials 2011; 32:5801-5809.
Hutton D L, Moore E M, Gimble J M, Grayson W L. Platelet-derived growth factor and spatiotemporal cues induce development of vascularized bone tissue by adipose-derived stem cells. Tissue Eng Part A 2013; 19:2076-2086.
Feinberg S E, Hollister S J, Halloran J W, Chu T M G, Krebsbach P H. Image-based biomimetic approach to reconstruction of the temporomandibular joint. Cells Tissues Organs 2001; 169:309-321.
Hollister S J, Levy R A, Chu T M, Halloran J W, Feinberg S E. An image-based approach for designing and manufacturing craniofacial scaffolds. Int J Oral Maxillofac Surg 2000; 29:67-71.
Alhadlaq A, Elisseeff J H, Hong L, Williams C G, Caplan A I, Sharma B, Kopher R A, Tomkoria S, Lennon D P, Lopez A, Mao J J. Adult stem cell driven genesis of human-shaped articular condyle. Ann Biomed Eng 2004; 32:911-923.
Weng Y L, Cao Y L, Silva C A, Vacanti M P, Vacanti C A. Tissue-engineered composites of bone and cartilage for mandible condylar reconstruction. J Oral Maxillofac Surg 2001; 59:185-190.
Alhadlaq A, Mao J J. Tissue-engineered neogenesis of human-shaped mandibular condyle from rat mesenchymal stem cells. J Dent Res 2003; 82:951-956.
Rivron N C, Raiss C C, Liu J, Nandakumar A, Sticht C, Gretz N, Truckenmuller R, Rouwkema J, van Blitterswijk C A. Sonic Hedgehog-activated engineered blood vessels enhance bone tissue formation. Proc Natl Acad Sci USA 2012; 109:4413-4418.
Amos P J, Kapur S K, Stapor P C, Shang H, Bekiranov S, Khurgel M, Rodeheaver G T, Peirce S M, Katz A J. Human adipose-derived stromal cells accelerate diabetic wound healing: impact of cell formulation and delivery. Tissue Eng Part A 2010; 16:1595-1606.
Cheng N C, Wang S, Young T H. The influence of spheroid formation of human adipose-derived stem cells on chitosan films on stemness and differentiation capabilities. Biomaterials 2012; 33:1748-1758.
Chen X, Aledia A S, Ghajar C M, Griffith C K, Putnam A J, Hughes C C, George S C. Prevascularization of a fibrin-based tissue construct accelerates the formation of functional anastomosis with host vasculature. Tissue Eng Part A 2009; 15:1363-1371.
Singh S, Wu B M, Dunn J C. Accelerating vascularization in polycaprolactone scaffolds by endothelial progenitor cells. Tissue Eng Part A 2011; 17:1819-1830.
Unger R E, Ghanaati S, Orth C, Sartoris A, Barbeck M, Halstenberg S, Motta A, Migliaresi C, Kirkpatrick C J. The rapid anastomosis between prevascularized networks on silk fibroin scaffolds generated in vitro with cocultures of human microvascular endothelial and osteoblast cells and the host vasculature. Biomaterials 2010; 31:6959-6967.
U.S. Patent Application Publication No. 20120035742 for “Methods, Devices and Systems for Bone Tissue Engineering Using a Bioreactor,” to Vunjak-Novakovic; G., et al., published Feb. 9, 2012.
U.S. Patent Application Publication No. 20120329156 for “Three-dimensional Scaffold and Method of Manufacturing the Same,” to Cho, D-W, et al., published Dec. 27, 2012.
U.S. Pat. No. 8,029,575 for “Porous and Nonporous Materials for Tissue Grafting and Repair,” to Borden, M., issued Oct. 4, 2011.
U.S. Pat. No. 7,494,811 for “In Vitro Growth of Tissues Suitable to the Formation of Bone and Bone Forming Tissue Formed Thereby.” to Wolfinbarger, L., Jr. and Qin; X., issued Feb. 24, 2009.
International PCT Patent Application Publication No. WO2006026730 for “Cultured Three Dimensional Tissues and Uses Thereof,” to Naughton, G., K., published Mar. 9, 2006.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A biodegradable scaffold for regenerating bone tissue, the scaffold configured to form a porous, three-dimensional (3D) network of interconnected void spaces, wherein the porous, three-dimensional network further comprises a hydrogel, and wherein the hydrogel comprises one or more cells encapsulated therein and one or more growth factors capable of promoting regeneration of bone tissue.

2. The scaffold of claim 1, wherein the scaffold comprises a biodegradable polymer selected from the group consisting of poly-ε-caprolactone (PCL), poly-lactic acid (PLA), poly-glycolic acid (PGA), and poly-lactic-co-glycolic acid (PLGA).

3. The scaffold of claim 2, wherein the biodegradable polymer comprises poly-ε-caprolactone (PCL).

4. The scaffold of claim 1, wherein the hydrogel comprises a natural polymer selected from the group consisting of fibrinogen, alginate, gelatin, collagen, hyaluronic acid (HA), chitosan, chondroitin sulfate, dextran sulfate, heparin, heparan sulfate, matrigel, and laminin.

5. The scaffold of claim 4, wherein the hydrogel comprises fibrinogen.

6. The scaffold of claim 1, wherein the one or more cells comprise autologous cells from bone marrow or adipose tissue.

7. The scaffold of claim 1, wherein the one or more cells comprise a stromal vascular fraction (SVF) comprising adipose-derived stromal/stem cells (ASCs) and endothelial cells.

8. The scaffold of claim 7, wherein the ASCs are substantially positive for mesenchymal markers CD73 and CD105.

9. The method of claim 7, wherein the ASCs are substantially negative for markers of the endothelial lineage, CD34+; VEGFR+; CD31+ and the endothelial cells are positive for markers of the endothelial linage.

10. The scaffold of claim 1, wherein the one or more cells are encapsulated in the hydrogel as mono-dispersed cells or as spheroid aggregates.

11. The scaffold of claim 1, wherein the one or more growth factors is selected from the group consisting of a platelet derived growth factor (PDGF), a transforming growth factor-β (TGF-β), a bone morphogenetic protein (BMP), a vascular endothelial cell growth factor (VEGF), an epithelial cell growth factor (EGF), and insulin-like growth factor (IGF).

12. The scaffold of claim 11, wherein the one or more growth factors comprises a platelet derived growth factor (PDGF).

13. The scaffold of claim 12, wherein the platelet derived growth factor (PDGF) comprises PDGF-BB.

14. The scaffold of claim 12, wherein the PDGF is present at a physiological concentration from about 0 ng/mL to about 20 ng/mL.

15. The method of claim 12, wherein the PDGF is present at a supraphysiological concentration from about 20 ng/mL to about 1000 ng/mL.

16. The scaffold of claim 1, wherein the growth factor promotes regeneration of bone tissue by enhancing vascular stability and osteogenic differentiation of the one or more cells.

17. The scaffold of claim 1, wherein the scaffold is configured to have an anatomical shape.

18. The scaffold of claim 17, wherein the anatomical shape is the shape of a mandible or a maxilla.

19. The scaffold of claim 1, wherein the biodegradable scaffold further comprises one or more vessels capable of delivering oxygen.

20. The scaffold of claim 1, wherein the biodegradable scaffold further comprises one or more factors capable of inducing osteogenic differentiation of stem cells.

21. The scaffold of claim 1, wherein the scaffold is formed by using three dimensional (3D)-printing.

22. The scaffold of claim 21, wherein the scaffold has about a 40% infill density.

23. A method for preparing a composite for promoting regeneration of bone tissue, the method comprising:
(a) providing a hydrogel;
(b) encapsulating one or more cells in the hydrogel;
(c) culturing the encapsulated cells for a first period of time in a vascular medium (VM);
(d) waiting for a second period of time, then culturing the encapsulated cells in an osteogenic medium (OM); and
(e) adding one or more growth factors to the hydrogel during at least one period of time while the encapsulated cells are cultured in either the VM or the OM.

24. The method of claim 23, wherein the hydrogel comprises a natural polymer selected from the group consisting of fibrinogen, alginate, gelatin, collagen, hyaluronic acid (HA), chitosan, chondroitin sulfate, dextran sulfate, heparin, heparan sulfate, matrigel, and laminin.

25. The method of claim 24, wherein the hydrogel comprises fibrinogen.

26. The method of claim 25, further comprising contacting the fibrinogen with thrombin to form a fibrin hydrogel.

27. The method of claim 23, wherein the one or more cells comprise autologous cells from bone marrow or adipose tissue.

28. The method of claim 23, wherein the one or more cells comprise a stromal vascular fraction (SVF) comprising adipose-derived stromal/stem cells (ASCs) and endothelial cells.

29. The method of claim 28, wherein the ASCs are substantially positive for mesenchymal markers CD73 and CD105.

30. The method of claim 28, wherein the ASCs are substantially negative for markers of the endothelial lineage, CD34+; VEGFR+; CD31+ and the endothelial cells are positive for markers of the endothelial linage.

31. The method of claim 23, wherein the one or more cells are encapsulated in the hydrogel as mono-dispersed cells or as spheroid aggregates.

32. The method of claim 23, wherein the one or more growth factors is selected from the group consisting of a platelet derived growth factor (PDGF), a transforming growth factor-β (TGF-β), a bone morphogenetic protein (BMP), a vascular endothelial cell growth factor (VEGF), an epithelial cell growth factor (EGF), and insulin-like growth factor (IGF).

33. The method of claim 32, wherein the growth factor comprises a platelet derived growth factor (PDGF).

34. The method of claim 33, wherein the platelet derived growth factor (PDGF) comprises PDGF-BB.

35. The method of claim 33, wherein the PDGF is present at a physiological concentration from about 0 ng/mL to about 20 ng/mL.

36. The method of claim 33, wherein the PDGF is present at a supraphysiological concentration from about 20 ng/mL to about 1000 ng/mL.

37. The method of claim 23, wherein the growth factor promotes regeneration of bone tissue by enhancing vascular stability and osteogenic differentiation of the one or more cells.

38. The method of claim 23, wherein the vascular medium (VM) comprises one or more components selected from the group consisting of Endothelial Basal Medium-2, FBS, penicillin/streptomycin, VEGF165, FGF-2, and 1 μg/mL L-ascorbic acid-2-phosphate.

39. The method of claim 23, wherein the osteogenic medium (OM) comprises one or more components selected from the group consisting of low glucose DMEM, FBS, and penicillin/streptomycin, and β-glycerophosphate and L-ascorbic acid-2-phosphate.

40. The method of claim 23, wherein the osteogenic medium (OM) further comprise one or more components selected from the group consisting of VEGF165, dexamethasone, and FGF-2.

41. The method of claim 23, further comprising adding one or more pro-inflammatory cytokines to the hydrogel.

42. The method of claim 41, wherein the one or more pro-inflammatory cytokines comprises IL-13 and tumor necrosis factor alpha (TNFα).

43. The method of claim 23, further comprising infusing the composite into a porous, three-dimensional biodegradable scaffold comprising a three-dimensional network of interconnected void spaces.

44. A method for treating a bone defect, the method comprising contacting the bone defect with a biodegradable scaffold for regenerating bone tissue, the scaffold configured to form a porous, three-dimensional (3D) network of interconnected void spaces, wherein the porous, three-dimensional network of further comprise a hydrogel, and wherein the hydrogel comprises one or more cells encapsulated therein and one or more growth factors capable of promoting regeneration of bone tissue.

45. The method of claim 44, wherein the bone defect comprises a critical-sized, non-healing bone defect.

46. The method of claim 44, wherein the bone defect comprises a bone loss arising from an event selected from the group consisting of a traumatic injury, a disease, surgery, natural aging, radiation, and a congenital defect.
